(19) United States
(12) Patent Application Publication
(10) Pub. No.: US 2010/0298402 A1
(43) Pub. Date: Nov. 25, 2010

(54) STILBENE DERIVATIVES AS PSTAT3/IL-6 INHIBITORS

(75) Inventors: Sridharan Rajagopal, Chennai (IN); Thangapazham Selvakumar, Chennai (IN); Kuppusamy Bharathimohan, Chennai (IN); Virendra Kachhadia, Chennai (IN); Sriram Rajagopal, Bangalore (IN); Rajendran Praveen, Corvallis, OR (US); Ramachandran Balaji, Bangalore (IN)

Correspondence Address: OLIFF & BERRIDGE, PLC, P.O. BOX 320850, ALEXANDRIA, VA, 22320-4850, US

(73) Assignee: ORCHID RESEARCH LABORATORIES LIMITED, Chennai (IN)

(21) Appl. No.: 12/734,484

(22) PCT Filed: Nov. 4, 2008
(86) PCT No.: PCT/IB2008/002943

§ 371 Date: Jul. 7, 2010

(30) Foreign Application Priority Data
Nov. 6, 2007 (IN) 2548/CHE/2007
May 13, 2008 (IN) 1165/CHE/2008

(51) Int. Cl. A61K 31/381; C07C 229/34; C07D 333/22; C07C 237/20; A61K 31/24; A61K 31/165; A61P 35/00; A61P 29/00; A61P 17/06; A61P 19/08; A61P 31/00; A61P 1/18; A61P 19/10; A61P 35/02; A61P 1/16; A61P 7/06; A61P 3/10; A61P 13/12; A61P 1/00; A61P 25/00

(52) U.S. Cl. 514/438; 560/42; 549/77; 564/165; 514/539; 514/620

(57) ABSTRACT

Described are novel compounds of the formula (I), their derivatives, analogs, tautomeric forms, stereoisomers, polymorphs, hydrates, solvates, intermediates, pharmaceutically acceptable salts, pharmaceutical compositions, metabolites, and prodrugs thereof. These novel compounds can inhibit pSTAT3/IL-6 and are useful as a therapeutic or ameliorating agent for diseases that are involved in cellular growth such as malignant tumors, autoimmune diseases, skin diseases, infections, inflammation, etc.

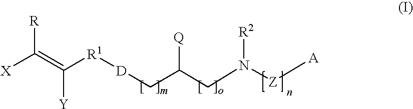

### FIELD

Described are novel compounds of the formula (I), their derivatives, analogs, tautomeric forms, stereoisomers which includes both geometrical and optical isomers; polymorphs, solvates, intermediates, pharmaceutically acceptable salts, pharmaceutical compositions, metabolites and prodrugs thereof.

Process for the preparation of the above said novel compounds of the formula (I) and pharmaceutical compositions containing these compounds is also described.

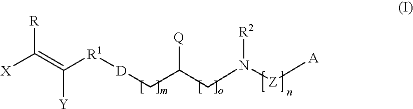

These compounds are inhibitors of pSTAT3/IL-6 and they can also arrest the cell growth in neoplastic cells, thereby inhibiting proliferation.

### BACKGROUND

The cell cycle deregulation and the molecular basis of cancer cell growth have been thoroughly exploited in the recent years. Inhibition of signal transduction has become a viable and attractive avenue in biomedical cancer research based on the discovery of a large number of somatic mutations in many different types of cancers that lead to deregulated growth signal transduction and subsequent aberrant growth, invasion, tumor-derived angiogenesis and metastasis. Most of the non-cytotoxic drugs that have been recently developed include protein kinase inhibitors such as Canertinib (irreversible Pan-erb B tyrosine kinase inhibitor), Dasatinib (dual inhibitor of Src and Abl kinases), Erlotinib (inhibitors of EGFR (erbB1 and HERD, Gefitinib (inhibitors of EGFR (erbB1 and HER1), Imatinib (BCR-ABL kinase inhibitors), Lapatinib (inhibitors of EGFR (erbB1 and erbB2), Sorafenib (multiple kinase inhibitors), Sunitinib (inhibitors of VEGFRR-2, PDGFR-β and FLT-3) and Vatalanib (VEGFR tyrosine kinase inhibitors). These types of orally active and relatively well-tolerated compounds can be used in the clinics; either as single or in combination with other well established cytotoxic agents.

Cytokines play an important role in the communication between cells of multicellular organisms. IL-6 promotes survival and proliferation of certain cancerous cell lines through the phosphorylation of STAT3. Inhibitors of JAK/STAT pathway likely represent potential therapeutic targets for cancer.

IL-6 has been found to be a growth factor for multiple myeloma cells; anti IL-6 antibodies were shown to block myeloma cell proliferation in leukemic patients. A need exists for a compound that blocks IL-6 mediated STAT3 activation at lower concentration and suppresses expression of proto-oncogene like C-myc, which is over expressed, rearranged or mutated in many malignancies.

One family of transcription factors responsible for transmitting a signal to a cell's nucleus are the proteins known as signal transducers and activators of transcription [for STATs see: Darwell et al. (1994), Science 264:1415; Ihle et al. (1994), Trends Biochem. Sci., 19:222; Ihle et al (1997), Current Opn. Cell Biol, 9: 233, N. Neamati, et. al. (2007) Current Cancer Drug Targets 7, 91-107)]. STATs are activated by contact with the phosphorylated cytokine receptors; activation results in the STAT polypeptides forming a dimer and entering the nucleus, where the STAT dimer binds to the regulatory region of a gene that is inducible by the particular cytokines. Binding of the activated STAT dimer triggers transcription of the gene. The STAT polypeptides (STAT1, STAT2, STAT4, STAT5a, STAT5b and STAT6) have molecular masses from 84-113 kDa. Each STAT protein contains a Src homology-2 (SH-2) domain capable of recognizing the cytoplasmic portion of the activated receptor (see Shuai et al. (1993) Nature 366:580). Additionally each cytokine receptor is specific for a particular STAT protein, and each STAT activates transcription of certain genes, thereby providing two layers of specificity in the cytokine induced signaling. STATs are proteins involved in signal transduction from cytokines and growth factor receptors. STAT3 has been demonstrated to up-regulate VEGF expression, which is necessary for angiogenesis and the maintenance of tumor vasculature. VEGF is produced at higher levels in, cancer cells and VEGF binds to a trans-membrane receptor tyrosine kinase on endothelial cells, activates endothelial-cell migration and proliferation to form new blood vessels. Blocking STAT3 signaling has been shown to inhibit Src and IL6 induced VEGF up regulation and might therefore also abrogate the induction of VEGF by other tyrosine kinase pathways that lie upstream. Unregulated activation of STAT3 and STATS has been demonstrated in a variety of tumor types, including breast carcinoma, prostate cancer, melanoma and leukemia among others. It has been reported that approximately 60% of breast tumors contains persistently activated STAT3 (Dechow et. al., Proc. Natl. Acad. Sci. USA 101, 10602, 2004). STAT3 as the molecular target and also various small molecule inhibitors of STAT3 were elucidated by Neamati, et. al (Current Cancer Drug Targets 2007, 7, 91-107 and J. Turkson, et. al., ACS Chemical Biology, 2007, 2, 787-798,). For example some of the molecules like AG490, WP1066, WP1130, S31-M2001, NSC74859, etc., were reported in the literature as STAT3 inhibitors and all are in the pre-clinical stage.

Recent studies report that resveratrol inhibits Src and STAT3 signaling which may induce apoptosis in malignant cells containing activated STAT3 protein. Resveratrol pre-treatment of endothelial cells significantly inhibits IL-6 induced STAT3 phosphorylation in both a dose and time dependent manner, suggesting resveratrol may regulate IL-6 induced ICAM-1 expression by inhibiting STAT3 phosphorylation (B. S. Wung. et. al., (2005). Life Sci. 64, 389-397).

WO2006060127 pertains to the invention of the compounds of formula A that possess inhibitory activity against β-adrenergic receptors and phosphodiesterases, including phosphodiesterase 3 (PDE3). The invention is further directed to novel compounds possessing both PDE-inhibitory and β-adrenergic receptor agonist activities

β-(Ar)ₙ-(L)ₘ-X A

wherein β represents —O—CH₂—CHOH—CH₂NZ₁Z₂. Z₁ and Z₂ are independently selected from a hydrogen radical and R¹ radicals. In yet further embodiments, Z₁ is hydrogen and Z₂ is C₁-C₄ alkyl. In yet further embodiments, Z₂ is isopropyl or t-butyl. R₁ is chosen from a hydrogen radical, C₁-C₆ alkyl radicals, C₁-C₆ cycloalkyl radicals, C₂-C₆ alkenyl radicals, C₂-C₆ cycloalkenyl radicals, and C₂-C₆ alkynyl radicals. Ar is chosen from aryl radicals and heteroaryl radicals; L is chosen from a direct bond, C₁-C₁₂ alkylene radicals, C₂-C₁₂ alkenylene radicals and C₂-C₁₂ alkynylene radicals, wherein one or more —CH₂— group(s) of the alkylene, alkenylene and alkynylene radicals is/are optionally replaced with —O—, —S—, —SO₂—, —NR₅—, C₃-C₈ cycloalkylene and/or C₃-C₅ heterocycloalkylene; the alkylene, alkenylene and alkynylene radicals are unsubstituted or substituted with one or more substituent(s) independently chosen from an oxo group and a hydroxyl group; m and n are integers chosen from 0 or 1. X is chosen from moieties of formula A-Y described therein.

### OBJECTIVES

Novel compounds of the general formula (I) are described. These compounds arrest cell growth in neoplastic cells, thereby inhibiting cell proliferation. STAT activation is dependent on tyrosine phosphorylation, which induces dimerization via, reciprocal phosphotyrosine (pTyr)-SH2 interactions between two STAT monomers and is a requirement for binding to specific DNA response elements. For example some of the molecules like AG490, WP1066, WP1130, S3I-M2001, NSC74859, etc., were reported in the literature as STAT inhibitors and all are in the pre-clinical stage. Because a variety of human cancers are associated with constitutively active pSTAT3, pSTAT3 represents an important target for cancer therapy.

### SUMMARY

Described are novel compounds of the general formula (I) as pSTAT3/IL-6 inhibitors,

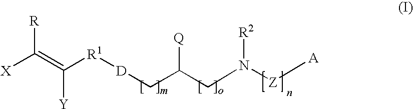

their derivatives, analogs, tautomeric forms, stereoisomers which includes both geometrical and optical isomers, polymorphs, solvates, intermediates, pharmaceutically acceptable salts, pharmaceutical compositions, metabolites and prodrugs thereof; wherein, the configuration around the double bonds may be E/Z and a mixture;

R and R¹ may be same or different and independently represent optionally substituted groups selected from cycloalkyl, aryl, arylalkyl, arylalkenyl, arylalkynyl, heterocyclyl, heteroaryl, heteroarylalkyl, heteroarylalkenyl and heteroarylalkynyl;

R² represents H, optionally substituted groups selected from alkyl, cycloalkyl, aryl and arylalkyl;

X and Y independently represent H; optionally substituted groups selected from alkyl, wherein when one of X or Y is hydrogen or unsubstituted alkyl, the other is neither of hydrogen nor of unsubstituted alkyl; —COOR³; —CONR³R⁴; —CN; —CH₂NR³R⁴; —CH₂—CH₂NR³R⁴; —CH₂OR³; —CH₂CH₂OR³; —CH₂OCONR³R⁴ and —CH₂NR³COR⁴; wherein R³ and R⁴ may be same or different and independently represent hydrogen; optionally substituted groups selected from alkyl; alkoxy; alkenyl; alkynyl; cycloalkyl; aryl; arylalkyl; arylalkenyl; arylalkynyl; heterocyclyl; heteroaryl; heteroarylalkyl; heteroarylalkenyl and heteroarylalkynyl or R³ and R⁴ can be combined to form a ring structure;

D represents —O— or —CH₂—;

Q represents H or OR⁵; wherein, R⁵ represents H, optionally substituted groups selected from alkyl, cycloalkyl, aryl, heterocyclyl and heteroaryl;

Z represents —CH₂— or —CO—;

A represents optionally substituted groups selected from aryl, arylalkyl, aryloxy, heterocyclyl and heteroaryl;

m, n and o are integers ranging from 0 to 5 and they may be same or different.

### DETAILED DESCRIPTION

Described are novel compounds of the general formula (I),

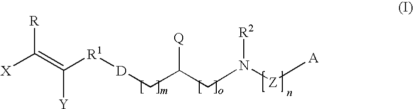

their derivatives, analogs, tautomeric forms, stereoisomers which includes both geometrical and optical isomers, polymorphs, solvates, intermediates, pharmaceutically acceptable salts, pharmaceutical compositions, metabolites and prodrugs thereof; wherein, the configuration around the double bonds may be E/Z and a mixture;

R and R¹ may be same or different and independently represent optionally substituted groups selected from cycloalkyl, aryl, arylalkyl, arylalkenyl, arylalkynyl, heterocyclyl, heteroaryl, heteroarylalkyl, heteroarylalkenyl and heteroarylalkynyl;

R² represents H, optionally substituted groups selected from alkyl, cycloalkyl, aryl and arylalkyl.

X and Y independently represent H; optionally substituted groups selected from alkyl, wherein when one of X or Y is hydrogen or unsubstituted alkyl, the other is neither of hydrogen nor of unsubstituted alkyl; —COOR^{S}; —CONR³R⁴; —CN; —CH₂NR³R⁴; —CH₂—CH₂NR³R⁴; —CH₂OR³; —CH₂CH₂OR³; —CH₂OCONR³R⁴ and —CH₂NR³COR⁴; wherein R³ and R⁴ may be same or different and independently represent hydrogen; optionally substituted groups selected from alkyl; alkoxy; alkenyl; alkynyl; cycloalkyl; aryl; arylalkyl; arylalkenyl; arylalkynyl; heterocyclyl; heteroaryl; heteroarylalkyl; heteroarylalkenyl and heteroarylalkynyl or R³ and R⁴ can be combined to form a ring structure;

D represents —O— or —CH₂—;

Q represents H or OR⁵; wherein R⁵ represents H, optionally substituted groups selected from alkyl, cycloalkyl, aryl, heterocyclyl and heteroaryl;

Z represents —CH₂— or —CO—; A represents optionally substituted groups selected from aryl, arylalkyl, aryloxy, heterocyclyl and heteroaryl;

m, n and o are integers ranging from 0 to 5 and they may be same or different;

when the groups R, R¹, R², R³, R⁴, R⁵ and A are substituted, the substituents which may be one or more are selected from halogens such as fluorine, chlorine, bromine and iodine; hydroxy; nitro; cyano; oxo (═O); thioxo (═S); azido; nitroso; amino; hydrazino; formyl; alkyl; alkoxy; aryl; haloalkyl groups such as trifluoromethyl, tribromomethyl and trichloromethyl; haloalkoxy groups such as —OCH₂Cl, —OCHF₂ and —OCF₃; arylalkoxy groups such as benzyloxy and phenylethoxy; cycloalkyl; —O-cycloalkyl; aryl; alkoxy; heterocyclyl; heteroaryl; alkylamino; —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a})SOR^{b}; —N(R^{a})SO₂R^{b}; —NR^{a}C(O)OR^{b}; —NR^{a}R^{b}; —NR^{a}C(O)R^{b}—; NR^{a}C(S)R^{b}—; —SONR^{a}R^{b}—; —SO₂NR^{a}R^{b}—; —OR^{a}; —OR^{a}C(O)OR^{b}—; —OC(O)NR^{a}R^{b}; OC(O)R^{a}; —OC(O)NR^{a}R^{b}—; —R^{a}NR^{b}R^{c}; —R^{a}OR^{b}—; —SR^{a}; —SOR^{a} and —SO₂R^{a}; R^{a}, R^{b} and R^{c} each independently represents hydrogen atom; substituted or unsubstituted groups selected from alkyl; aryl; arylalkyl; cycloalkyl; heterocyclyl; heteroaryl and heteroarylalkyl and R^{a}, R^{b} and R^{c} are combined to form 3-7 membered ring having 0-2 hetero atoms.

The substituents are in turn are further substituted by halogens such as fluorine, chlorine, bromine and iodine; hydroxy; nitro; cycloalkyl; cyano; azido; nitroso, amino, hydrazino, formyl; alkyl and haloalkyl groups, such as trifluoromethyl and tribromoethyl;

Furthermore, whenever the groups R, R¹, R², R³, R⁴, R⁵, R^{a}, R^{b}, R^{c} and A represent ring systems, the ring systems may be substituted or unsubstituted monocyclic or polycyclic, partially saturated or aromatic containing 1 to 4 heteroatoms selected from O, S or N.

The term “alkyl” refers to straight or branched aliphatic hydrocarbon groups having the specified number of carbon atoms, which are attached to the rest of the molecule by a single atom. Preferred alkyl groups include, without limitation, methyl, ethyl, n-propyl, isopropyl, butyl, isobutyl, t-butyl, pentyl, hexyl, heptyl, octyl and the like.

The term “aryl” refers to aromatic radicals having 6 to 14 carbon atoms, which may be optionally substituted by one or more substituents. Preferred aryl groups include, without limitation, phenyl, naphthyl, indanyl, biphenyl and the like. Substituted or unsubstituted arylene groups such as phenylene, biphenylene, naphthylene, anthracenylene, phenanthrylene, indanylene and the like.

The term “arylalkyl” refers to an aryl group directly bonded to an alkyl group, which may be optionally substituted by one or more substituents. Preferred arylalkyl groups include, without limitation, —CH₂C₆H₅, —C₂H₄C₆H₅ and the like.

The term “heterocyclyl” refers to a stable 3 to 15 membered rings radical, which consists of carbon atoms and from one to five heteroatoms selected from nitrogen, phosphorus, oxygen and sulfur. For purposes of this invention the heterocyclic ring radical may be monocyclic, bicyclic or tricyclic ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated. Preferred heterocyclyl groups include, without limitation, azetidinyl, acridinyl, benzodioxolyl, benzodioxanyl, benzopyranyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, decahydroisoquinolyl, benzimidazolyl, thiadiazolyl, benzopyranyl, benzothiazolyl, benzooxazolyl, thienyl, morpholinyl, thiomorpholinyl, thiamorpholinyl sulfoxide, furyl, tetrahydrofuryl, tetrahydropyranyl, chromanyl and isochromanyl. The heterocyclyl ring radical may be attached to the main structure at any heteroatom or carbon atom that results in the creation of a stable structure.

The term “heteroaryl” refers to an aromatic heterocyclic ring radical as defined above. The heteroaryl ring radical may be attached to the main structure at any heteroatom or carbon atom that results in the creation of a stable structure.

The term “heteroarylalkyl” refers to a heteroaryl ring radical as defined above directly bonded to an alkyl group. The heteroarylalkyl radical may be attached to the main structure at any carbon atom from an alkyl group.

The term “cycloalkyl” refers to non-aromatic mono or polycyclic ring systems of about 3 to 12 carbon atoms. Preferred cycloalkyl groups include, without limitation, cyclopropyl, cyclobutyl, cyclopentyl, cyclohexyl, cyclooctanyl and the like; preferred polycyclic rings include, without limitation, perhydronaphthyl, adamantyl and norbornyl groups, bridged cyclic groups or spirobicyclic groups e.g. Spiro[4.4]-non-2-yl and the like.

The term “alkenyl” refers to an aliphatic hydrocarbon group containing a carbon-carbon double bond and which may be straight or branched chain having about 2 to 10 carbon atoms, which may be optionally substituted by one or more substituents. Preferred alkenyl groups include, without limitation, ethenyl, 1-propenyl, 2-propenyl, iso-propenyl, 2-methyl-1-propenyl, 1-butenyl, 2-butenyl and the like.

The term “arylalkenyl” refers to an aromatic ring radical directly bonded to an alkenyl group. The aryl radical may be attached to the main structure at any carbon from the alkenyl group. Preferred arylalkenyl groups include, without limitation, phenylethenyl, phenylpropenyl and the like.

The term “heteroarylalkenyl” refers to a heteroaryl ring radical directly bonded to an alkenyl group. The heteroaryl radical may be attached to the main structure at any carbon from the alkenyl group. Preferred heteroarylalkenyl groups include, without limitation, thienylpropenyl, indolylpropenyl, pyridinylethenyl and indolypropenyl.

The term “alkylthio” refers to an alkyl group attached via a sulfur linkage to the rest of the molecule, which may be optionally substituted by one or more substituents. Preferred alkylthio groups include, without limitation, —SCH₃, —SC₂H₅ and the like.

The term “alkoxy” refers to an alkyl group attached via an oxygen linkage to the rest of the molecule. Preferred alkoxy groups include, without limitation, —OCH₃, —OC₂H₅ and the like.

The term “aryloxy” refers to an aryl group attached via an oxygen linkage to the rest of the molecule. Preferred aryloxy groups include, without limitation, —O-phenyl, —O-biphenyl and the like.

The term “alkylamino” refers to an alkyl group as defined above attached via an amino linkage to the rest of the molecule. Preferred alkylamino groups include, without limitation, —NHCH₃, —N(CH₃)₂ and the like.

The term “alkynyl” refers to straight or branched hydrocarbyl radicals having at least one carbon-carbon triple bond and having in the range of 2-12 carbon atoms. Preferred alkynyl groups include, without limitation, ethynyl, propynyl, butynyl and the like.

The term “arylalkynyl” refers to an aromatic ring radical directly bonded to an alkynyl group. The aryl radical may be attached to the main structure at any carbon atom from the alkynyl group.

The term “heteroarylalkynyl” refers to a heteroaryl radical directly bonded to an alkynyl group. The heteroaryl radical may be attached to the main structure at any carbon atom from the alkynyl group.

Furthermore, the compound of formula (I) can be its derivatives, analogs, tautomeric forms, stereoisomers, geometrical isomers, polymorphs, solvates, intermediates, pharmaceutically acceptable salts, pharmaceutical compositions, metabolites and prodrugs.

Pharmaceutically acceptable solvates may be hydrates or comprising of other solvents of crystallization such as alcohols.

The compounds described herein can be either in E or Z geometrical isomers and in some cases mixtures can also be present. In cases where two or more double bonds are present in formula (I), can give rise to more than two geometrical isomers and in these cases the invention is said to cover all the isomers.

It is understood that included in the family of compounds of formula (I) are isomeric forms including tautomers and stereoisomers (diastereoisomers, enantiomers and geometrical isomers in “E” or “Z” configurational isomer or a mixture of E and Z isomers). It is also understood that some isomeric forms such as diastereomers, enantiomers and geometrical isomers can be separated by physical and/or chemical methods and by those skilled in the art.

Compounds disclosed herein may exist as single stereoisomers, racemates and or mixtures of enantiomers and/or diastereomers. All such single stereoisomers, racemates and mixtures thereof are intended to be within the scope of the subject matter described.

The phrase “pharmaceutically acceptable” refers to compounds or compositions that are physiologically tolerable and do not typically produce allergic or similar untoward reaction, including but are not limited to, gastric upset or dizziness when administered to mammal.

Pharmaceutically acceptable salts include salts derived from inorganic bases such as like Li, Na, K, Ca, Mg, Fe, Cu, Zn and Mn; salts of organic bases such as N,N′-diacetylethylenediamine, glucamine, triethylamine, choline, dicyclohexylamine, benzylamine, trialkylamine, thiamine, guanidine, diethanolamine, α-phenylethylamine, piperidine, morpholine, pyridine, hydroxyethylpyrrolidine, hydroxyethylpiperidine and the like, ammonium, substituted ammonium salts, aluminum salts. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates and ketoglutarates.

Described herein are prodrugs of the compound of formula (I), which on administration undergoes chemical conversion by metabolic processes before becoming active pharmacological substances. In general, such prodrugs will be functional derivatives of a compound of the invention, which are readily convertible in vivo into a compound of the invention.

“Prodrug” means a compound, which is convertible in vivo by metabolic means (that is by hydrolysis, reduction or oxidation) to a compound of formula (I). For example an ester prodrug of a compound of formula (I) containing hydroxyl group may be convertible by hydrolysis in vivo to the parent molecule.

The active compounds disclosed can also be prepared in any solid or liquid physical form, for example the compound can be in a crystalline form, in amorphous form and have any particle size. Furthermore, the compound particles may be micronized or nanoized, or may be agglomerated, particulate granules, powders, oils, oily suspensions or any other form of solid or liquid physical forms.

Described herein are also pharmaceutical compositions, containing one or more of the compounds of the general formula (I) as defined above, their derivatives, analogs, tautomeric forms, stereoisomers, polymorphs, hydrates, metabolites, prodrugs, pharmaceutically acceptable salts, pharmaceutically acceptable solvates in combination with the usual pharmaceutically employed carriers, diluents and the like, useful for the treatment of and/or proliferative disorders.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. The compositions may be prepared by processes known in the art. Suitable pharmaceutically acceptable carriers include solid fillers or diluents and sterile aqueous or organic solutions. The active compound will be present in such pharmaceutical compositions in the amounts sufficient to provide the desired dosage in the range as described above. Suitable routes of administration include systemic, such as orally or by parenteral administration such as subcutaneous, intramuscular, intravenous and intradermal routes. Thus for oral administration, the compounds can be combined with a suitable solid or liquid carrier or diluent to form capsules, tablets, powders, syrups, solutions, suspensions and the like. The pharmaceutical compositions, may, if desired, contain additional components such as flavorants, sweeteners, excipients and the like. For parenteral administration, the compounds can be combined with a sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously or intramuscularly.

The compounds of formula (I) can also be administered as a pharmaceutical composition in a pharmaceutically acceptable carrier, preferably formulated for oral administration.

The compounds described herein may also exhibit polymorphism. This invention further includes different polymorphs of the compounds. The term polymorph refers to a particular crystalline state of a substance, having particular physical properties such as X-ray diffraction, IR spectra, melting point and the like.

This invention, in addition to the above listed compounds, is intended to encompass the use of homologs and analogs of such compounds. In this context, homologs are molecules having substantial structural similarities to the above-described compounds and analogs are molecules having substantial biological similarities regardless of structural similarities.

Compounds of the invention having the capability to modulate (e.g., reduce or eliminate) signaling of the STAT3 and/or STATS signaling pathway in vitro and/or in vivo, or to inhibit the growth of cancer cells in vitro and/or in vivo by inhibition of STAT3 and/or STATS signaling or a different mechanism, would be considered to have the desired biological activity in accordance with the subject invention. For therapeutic applications, compounds of the subject invention have the capability to inhibit activation of the STAT3 and/or STAT5 signaling pathway, or to inhibit the growth of cancer cells in vitro and/or in vivo by inhibition of STAT3 and/or STAT5 signaling or a different mechanism.

One aspect of the subject invention provides methods for using the compounds of the invention as STAT3 inhibitors and/or as anti-proliferative agents.

Another aspect of the invention is related to the use of the compounds in inhibitory IL-6 production. Compounds of the invention can be used to inhibit both IL-6 production and STAT3 activation.

One aspect of the invention concerns a method of treating a proliferation disorder in a subject, comprising administering an effective amount of at least one compound of the invention to the subject. In one embodiment, the disorder is mediated by cells harboring constitutively active STAT3.

Another aspect of the invention concerns a method of suppressing the growth or apoptosis of malignant cells, comprising contacting the cells in vitro or in vivo with an effective amount of at least one compound of the invention. In one embodiment, the malignant cells are mediated by cells harboring constitutively active STAT3.

Another aspect of the invention concerns a method of inhibiting constitutive activation of STAT3 in cells, preventing STAT3 dimerization in mammalian cells or disrupting STAT3-DNA binding.

The methods of the present invention can be advantageously combined with at least one additional treatment method, including but not limited to, chemotherapy, radiation therapy, or any other therapy known to those of skills in the art for the treatment and management of proliferation disorders such as cancer.

The terms “Cancer” and “Cancerous” refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

The term “tumor” refers to all neoplastic cell growth and proliferation, whether malignant or benign and all pre-cancerous, cancerous cells and tissues.

The invention also provides a method of treatment of cancer in patients including administration of a therapeutically effective amount of a compound formula (I).

The present invention provides a method of treatment of a disorder caused by, associated with or accompanied by disruptions of cell proliferation and/or angiogenesis including administration of a therapeutically effective amount of a compound of formula (I).

The disorder is either a proliferative disorder or is selected from the group consisting of but is not limited to, cancer, inflammatory diseases/immune disorder, fibrotic diseases (e.g. liver fibrosis), diabetes, autoimmune disease, chronic and acute neurodegenerative disease, Huntington's disease and infectious disease.

The compounds described herein are used in the treatment or prevention of cancer. The cancer can include solid tumors or hematologic malignancies.

The invention provides a method of treatment of cancer in patients including administration of effective amount of formula (I). The cancer can be either a hematologic malignancy and this form of malignancy is selected from the group consisting of B-cell lymphoma, T-cell lymphoma and leukemia. In the case of solid tumors, the tumors are selected from the group consisting of breast cancer, lung cancer, ovarian cancer, prostate cancer, head cancer, neck cancer, renal cancer, gastric cancer, colon cancer, pancreatic cancer and brain cancer.

The term “therapeutically effective amount” or “effective amount” is an amount sufficient to effect beneficial or desired results. An effective amount can be administered in one or more administrations. An effective amount is typically sufficient to palliate, ameliorate, stabilize, reverse, slow or delay the progression of the disease state.

In another aspect, the compound may be administered in combination therapy by combining the compound of formula (I) with one or more separate agents, not limited to targets such as HDAC, DNA methyltransferase, heat shock proteins (e.g. HSP90) kinase and other matrix metalloproteinases.

“Combination therapy” includes the administration of the subject compounds in further combination with other biologically active ingredients (such as, but are not limited to, different antineoplastic agents) and non-drug therapies (such as, but are not limited to, surgery or radiation treatment). The compounds described herein can be used in combination with other pharmaceutically active compounds, preferably, which will enhance the effect of the compounds of the invention. The compounds can be administered simultaneously or sequentially to the other drug therapy.

In another aspect, the subject compounds may be combined with the antineoplastic agents (e.g. small molecules, monoclonal antibodies, antisense RNA and fusion proteins) that inhibit one or more biological targets. Such a combination may enhance therapeutic efficacy over the efficacy achieved by any of the agents alone and may prevent or delay the appearance of resistant variants.

The compounds of the invention are administered in combination with chemotherapeutic agents. Chemotherapeutic agents consist of a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to cancer or its treatment.

The term “subject” as used herein is meant to include all mammals, and in particular humans, in need of treatment. The therapeutically effective amount will vary depending upon the subject and disease condition being treated, the weight and age of the subject, the severity of the disease condition, the particular compound of formula (I) chosen, the dosing regimen to be followed, timing of administration, the manner of administration and the like, all of which can readily be determined by one of ordinary skill in the art.

A term once described, the same meaning applies for it, throughout the patent.

Representative compounds include:
1. 2-{4-[3-(2,4-Dimethoxybenzylamino)-2-hydroxypropoxy]phenyl}-3-(4-fluoro-3-methylphenyl)acrylic acid methyl ester;
2. 2-{4-[3-(2,4-Dimethoxybenzylamino)-2-hydroxypropoxyl]phenyl}-3-(4-fluorophenyl)acrylic acid methyl ester;
3. 2-{4-[3-(2,4-Dimethoxybenzylamino)-2-hydroxypropoxyl]phenyl}-3-(3,5-dimethoxyphenyl)acrylic acid methyl ester;
4. 2-{4-[3-(4-Trifluoromethoxybenzylamino)-2-hydroxypropoxyl]phenyl}dimethoxyphenyl)acrylic acid methyl ester;
5. 2-{4-[3-(2-Methoxyphenoxyethylamino)-2-hydroxypropoxyl]phenyl}-3-(3,5-dimethoxyphenyl)acrylic acid methyl ester;
6. 2-{4-[3-(2,4-Dimethoxy benzylamino)-2-hydroxypropoxyl]phenyl}-3-(3,4,5-trimethoxyphenyl)acrylic acid methyl ester;
7. 2-{4-[3-(2-Methoxyphenoxyethylamino)-2-hydroxypropoxyl]phenyl}-3-(4-fluoro-3-methoxyphenyl)acrylic acid methyl ester;
8. 2-{4-[3-(4-Difluoromethoxybenzylamino)-2-hydroxypropoxyl]phenyl}-3-(3,4,5-trimethoxyphenyl)acrylic acid methyl ester;
9. 2-{4-[3-(2,4-Dimethoxybenzylamino)-2-hydroxypropoxyl]phenyl}-3-(4-fluoro-3-methoxyphenyl)acrylic acid methyl ester;
10. 3-{4-[3-(4-Fluorobenzylamino)-2-hydroxypropoxyl]phenyl}-2-phenylacrylic acid methyl ester;
11. 2-{4-[3-(2-Methoxyphenoxyethylamino)-2-hydroxypropoxyl]phenyl}-3-(3,4,5-trimethoxyphenyl)acrylic acid methyl ester;
12. 3-{4-[3-(2,4-Dimethoxybenzylamino)-2-hydroxypropoxyl]phenyl}-2-(4-methoxyphenyl)acrylic acid methyl ester;
13. 2-{4-[3-(2,4-Dimethoxy benzylamino)-2-hydroxypropoxyl]phenyl}-3-(3,4,-dimethoxyphenyl)acrylic acid methyl ester;
14. 2-{4-[3-(2-Methoxyphenoxyethylamino)-2-hydroxypropoxyl]phenyl}-3-(3,4,-dimethoxyphenyl)acrylic acid methyl ester;
15. 2-{4-[3-(2,4-Dimethoxy benzylamino)-2-hydroxypropoxyl]phenyl}-3-(3,4-difluorophenyl)acrylic acid methyl ester;
16. 3-{4-[3-(2,4-Dimethoxy benzylamino)-2-hydroxypropoxyl]phenyl}-2-phenyl acrylic acid methyl ester;
17. 2-{4-[3-(2,4-Dimethoxybenzylamino)-2-hydroxypropoxy]phenyl}-3-phenylacrylic acid methyl ester;
18. 2-{4-[3-(Benzylamino)-2-hydroxypropoxy]phenyl}-3-(3,5-dimethoxyphenyl)acrylic acid methyl ester;
19. 2-{4-[3-(2-Methoxyphenoxyethylamino)-2-hydroxypropoxy]phenyl}-3-(4-fluoro-3-methylphenyl)acrylic acid methyl ester;
20. 2-{4-[3-(4-Fluorobenzylamino)-2-hydroxypropoxy]phenyl}-3-(3,5-dimethoxyphenyl)acrylic acid methyl ester;
21. 2-{4-[3-(Benzylamino)-2-hydroxypropoxy]phenyl}-3-(4-fluoro-3-methylphenyl)acrylic acid methyl ester;
22. 2-{4-[3-(2,4-Dimethoxybenzylamino)-2-hydroxypropoxy]phenyl}-3-(4-chlorophenyl)acrylic acid methyl ester;
23. 2-{4-[3-(2-Methoxyphenoxy-ethylamino)-2-hydroxy-propoxy]-phenyl}-3-(4-(methylthio)phenyl)-acrylic acid methyl ester;
24. 2-{4-[3-(Benzylamino)-2-hydroxypropoxy]phenyl}-3-phenylacrylic acid methyl ester;
25. 2-{4-[3-(2-Methoxyphenoxyethylamino)-2-hydroxypropoxy]phenyl}-3-phenylacrylic acid methyl ester;
26. 2-{4-[3-(2-Methoxyphenoxyethylamino)-2-hydroxypropoxy]phenyl}-3-(4-fluorophenyl)acrylic acid methyl ester;
27. 2-{4-[3-(Benzylamino)-2-hydroxypropoxy]phenyl}-3-(4-chlorophenyl)acrylic acid methyl ester;
28. 2-{4-[3-(2,4-Dimethoxybenzylamino)-2-hydroxypropoxy]phenyl}-3-(4-(methylthio)phenyl)acrylic acid methyl ester;
29. 3-{4-[3-(2-Methoxyphenoxyethylamino)-2-hydroxypropoxy]phenyl}-2-(4-(methoxy)phenyl)acrylic acid methyl ester;
30. 3-{4-[3-(Benzylamino)-2-hydroxypropoxy]phenyl}-2-(4-(methoxy)phenyl)acrylic acid methyl ester;
31. 3-{4-[3-(Benzylamino)-2-hydroxypropoxy]phenyl}-2-(thiophen-2-yl)acrylic acid methyl ester;
32. 2-{4-[3-(2,4-Dimethoxybenzylamino)-2-hydroxypropoxy]phenyl}-3-(2,4-dimethoxyphenyl)acrylic acid methyl ester;
33. 2-{4-[3-(2-Methoxyphenoxyethylamino)-2-hydroxypropoxy]phenyl}-3-(2,4-dimethoxyphenyl)acrylic acid methyl ester;
34. 3-{4-[3-(2-Methoxyphenoxyethylamino)-2-hydroxypropoxy]phenyl}-2-(thiophen-2-yl)acrylic acid methyl ester;
35. 3-{4-[3-(2,4-Dimethoxybenzylamino)-2-hydroxypropoxy]phenyl}-2-(4-fluorophenyl)acrylic acid methyl ester;
36. 3-{4-[3-(Benzylamino)-2-hydroxypropoxy]phenyl}-2-(4-fluorophenyl)acrylic acid methyl ester;
37. 3-{4-[3-(2,4-Dimethoxybenzylamino)-2-hydroxypropoxy]phenyl}-2-(4-methylphenyl)acrylic acid methyl ester;
38. 3-{4-[3-(2-Methoxyphenoxyethylamino)-2-hydroxypropoxy]phenyl}-2-(4-methylphenyl)acrylic acid methyl ester;
39. 3-{4-[3-(Benzylamino)-2-hydroxypropoxy]phenyl}-2-(4-methylphenyl)acrylic acid methyl ester;
40. 3-{4-[3-(2,4-Dimethoxybenzylamino)-2-hydroxypropoxy]phenyl}-2-(thiophen-2-yl)acrylic acid methyl ester;
41. 3-{4-[3-(2-Methoxyphenoxyethylamino)-2-hydroxypropoxy]phenyl}-2-(4-fluorophenyl)acrylic acid methyl ester;
42. 3-{4-[3-(2-Methoxyphenoxyethylamino)-2-hydroxypropoxy]phenyl}-2-phenylacrylic acid methyl ester;
43. 3-{2-[3-(Benzylamino)-2-hydroxypropoxy]phenyl}-2-(4-methoxyphenyl)acrylic acid methyl ester;
44. 3-{4-[3-(Benzylamino)-2-hydroxypropoxy]phenyl}-2-phenylacrylic acid methyl ester;
45. 3-{2-[3-(2,4-Dimethoxybenzylamino)-2-hydroxypropoxy]phenyl}-2-(4-methoxy phenyl)acrylic acid methyl ester;
46. 3-{2-[3-(2-Methoxyphenoxyethylamino)-2-hydroxypropoxy]phenyl}-2-(4-methoxyphenyl)acrylic acid methyl ester;
47. 3-{4-[3-(2,4-Dimethoxybenzylamino)-2-hydroxypropoxy]phenyl}-2-(3-chloro phenyl)acrylic acid methyl ester;
48. 3-{4-[3-(2-Methoxyphenoxyethylamino)-2-hydroxypropoxy]phenyl}-2-(3-chloro phenyl)acrylic acid methyl ester;
49. 3-{4-[3-(Benzylamino)-2-hydroxypropoxy]phenyl}-2-(3-chlorophenyl)acrylic acid methyl ester;
50. 3-{4-[3-(2,4-Dimethoxybenzylamino)-2-hydroxypropoxy]phenyl}-2-(3-fluoro phenyl)acrylic acid methyl ester;
51. 3-{4-[3-(2-Methoxyphenoxyethylamino)-2-hydroxypropoxy]phenyl}-2-(3-fluoro phenyl)acrylic acid methyl ester;
52. 3-{4-[3-(2,4-Dimethoxybenzylamino)-2-hydroxypropoxy]phenyl}-2-(2-chlorophenyl)acrylic acid methyl ester
53. 3-{4-[3-(2,4-Dimethoxybenzylamino)-2-hydroxypropoxy]phenyl}-2-(2-fluoro phenyl)acrylic acid methyl ester;
54. 3-{4-[3-(2-Methoxyphenoxyethylamino)-2-hydroxy-propoxy]phenyl}-2-(2-fluoro phenyl)acrylic acid methyl ester;
55. 3-{4-[3-(Benzylamino)-2-hydroxypropoxy]phenyl}-2-(2-fluorophenyl)acrylic acid methyl ester;
56. 2-{4-[3-(2,4-Dimethoxybenzylamino)-2-hydroxypropoxy]phenyl}-3-(5-methylthiophen-2-yl)acrylic acid methyl ester;
57. 2-{4-[3-(2-Methoxyphenoxyethylamino)-2-hydroxypropoxy]phenyl}-3-(5-methylthiophen-2-yl)acrylic acid methyl ester;
58. 3-{4-[3-(Benzylamino)-2-hydroxypropoxy]phenyl}-2-(3-fluorophenyl)acrylic acid methyl ester;
59. 3-{4-[3-(2-Methoxyphenoxyethylamino)-2-hydroxypropoxy]phenyl}-2-(2-chloro phenyl)acrylic acid methyl ester;
60. 3-{4-[3-(Benzylamino)-2-hydroxypropoxy]phenyl}-2-(2-chlorophenyl)acrylic acid methyl ester;
61. 2-{4-[3-(Benzylamino)-2-hydroxypropoxy]phenyl}-3-(5-methylthiophen-2-yl)acrylic acid methyl ester;
62. 2-{4-[3-(2,4-Dimethoxybenzylamino)-2-hydroxypropoxy]phenyl}-3-(2,4-dimethoxyphenyl)acrylamide;
63. 2-{4-[3-(2,4-Dimethoxybenzylamino)-2-hydroxypropoxy]phenyl}-3-(3,5-dimethoxyphenyl)acrylamide;
64. 2-{4-[3-(2,4-Dimethoxybenzylamino)-2-hydroxypropoxy]phenyl}-3-(3,4-difluorophenyl)acrylamide;
65. 2-{4-[3-(2-Methoxyphenoxyethylamino)-2-hydroxypropoxy]phenyl}-3-(3,4-difluorophenyl)acrylamide;
66. 2-{4-[3-(2,4-Dimethoxybenzylamino)-2-hydroxypropoxy]phenyl}-3-(4-fluorophenyl)acrylamide;
67. 2-{4-[3-(2-Methoxyphenoxyethylamino)-2-hydroxypropoxy]phenyl}-3-(4-fluorophenyl)acrylamide;
68. 2-{4-[3-(2-Methoxyphenoxyethylamino)-2-hydroxypropoxy]phenyl}-3-(2,4-dimethoxyphenyl)acrylamide;
69. 2-{4-[3-(Benzylamino)-2-hydroxypropoxy]phenyl}-3-(3,4-difluorophenyl)acrylamide;
70. 2-{4-[3-(2-Methoxyphenoxyethylamino)propoxy]phenyl}-3-(3,5-dimethoxyphenyl)acrylic acid methyl ester;
71. 2-{4-[3-(2,4-dimethoxybenzylamino)propoxy]phenyl}-3-(3,5-dimethoxyphenyl)acrylic acid methyl ester and
72. 2-{4-[3-(Benzylamino)propoxy]phenyl}-3-(3,5-dimethoxyphenyl)acrylic acid methyl ester.

According to another feature of the present invention, there is provided a process as shown in the following schemel, for the preparation of compounds of the formula (I), wherein all the groups R, R¹, R², A, X, Y, Q, Z, m, n and o are as defined earlier and when one of X or Y is hydrogen or unsubstituted alkyl, the other is neither of hydrogen nor of unsubstituted alkyl.

The said process comprises the steps of:
1. Condensing the compound of formula (Ia) with the compound of formula (Ib) or condensing the compound of formula (IA) with the compound of formula (IB) in the presence of Ac₂O (acetic anhydride) and an organic base to yield a compound, which on subsequent hydrolysis gives a hydroxy acid compound, that on further reaction in MeOH and catalytic amounts of H₂SO₄ or with an acid activating agent such as EDCI (1-ethyl-3-(3-dimethylaminopropyl)carbodiimide hydrochloride), HOBt (N-hydroxybenzotriazole) or by converting the acid to acid chloride using oxalyl chloride and the like in the presence of an amino compound or with the alcohol compound to give the corresponding amide or ester of formula (Ic).
2. Reacting the compound of formula (Ic) with epichlorohydrin in refluxing IPA (isopropanol) or DMF/K₂CO₃ to give compound of formula (Id).
3. Reacting the compound of formula (Id) with the corresponding amino compound in IPA/EtOAc (ethyl acetate) to give the compound of formula (I).
4. Reacting the compound of formula (Ic) with L-alkane-L, for example a dihaloalkanes such as dibromoalkane in the presence of a base to give the compound of formula (Ie), wherein L is a good leaving group, such as halogen, tosylate, mesylate, besylate and the like.
5. Reacting the compound of formula (Ie) with the corresponding amino compound in DMF to give the compound of formula (I).

In any of the reactions mentioned below, any reactive group in the substrate molecule may be protected according to the conventional chemical practice. Suitable protecting groups in any of the above-mentioned reactions are those used conventionally in the art. The methods of formation and removal of such protecting groups are those conventional methods appropriate to the molecule being protected.

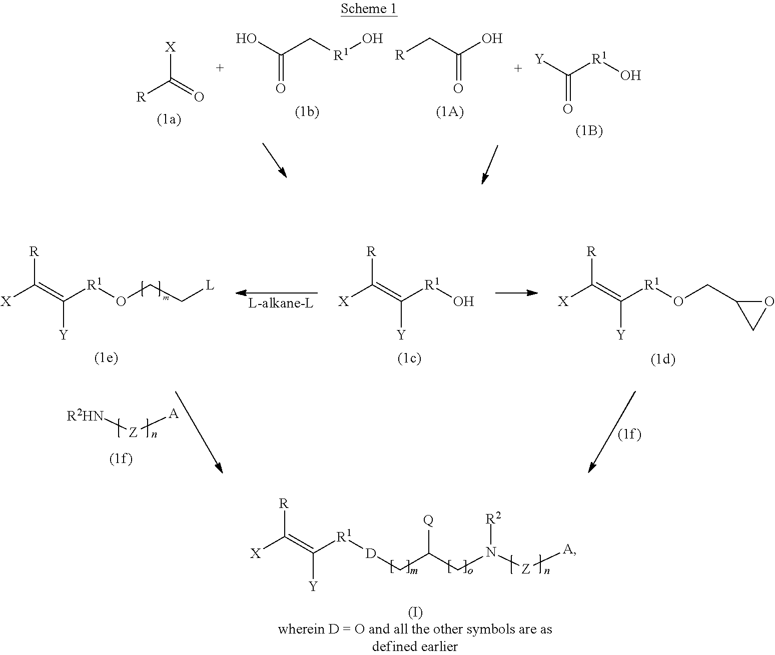

The pharmaceutically acceptable salts are prepared by reacting the compound of formula (I) with 1 to 4 equivalents of a base such as sodium hydroxide, sodium methoxide, sodium hydride, potassium t-butoxide, and calcium hydroxide, magnesium hydroxide and the like, in solvents like ether, THF (tetrahydrofuran), methanol, t-butanol, dioxane, isopropanol, ethanol etc. Mixtures of solvents may be used. Organic bases like lysine, arginine, diethanolamine, choline, guanidine and their derivatives etc. may also be used. Alternatively, acid addition salts are prepared by treatment with acids such as hydrochloric acid, hydrobromic acid, nitric acid, sulfuric acid, phosphoric acid, p-toluenesulfonic acid, methanesulfonic acid, acetic acid, citric acid, maleic acid, salicylic acid, hydroxynaphthoic acid, ascorbic acid, palmitic acid, succinic acid, benzoic acid, benzene sulfonic acid, tartaric acid and the like in solvents like ethyl acetate, ether, alcohols, acetone, THF (tetrahydrofuran), dioxane etc. Mixture of solvents may also be used.

The invention is explained in details in the examples given below which are provided by the way of illustration only and therefore should not be construed to limit the scope of the invention.

### Example 1

### Synthesis of 2-{4-[3-(2,4-dimethoxybenzylamino)-2-hydroxypropoxy]phenyl}-3-(4-fluoro-3-methylphenyl)acrylic acid methyl ester

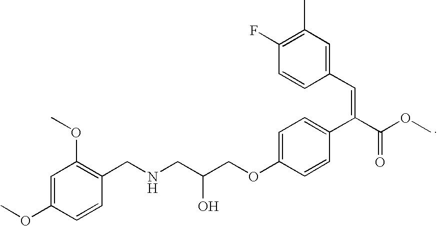

### Step-1

### Preparation of 2-[4-(acetyloxy)phenyl]-3-(3-methyl-4-fluorophenyl)acrylic acid

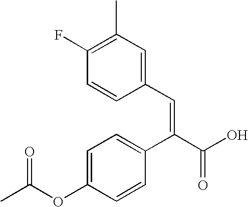

A mixture of 3-methyl-4-fluorobenzaldehyde (2 g, 14.4 mmol) and 4-hydroxyphenylacetic acid (2.2 g, 14.4 mmol) was dissolved under stirring with acetic anhydride (8 mL). DIPEA (diisopropylethyl amine) (6.5 mL, 50.5 mmol) was added to the reaction mixture dropwise and stirred for 12 hours. Upon completion (as monitored by TLC using 1:1 hexane:ethyl acetate as eluent), the reaction mixture was diluted with water (150 mL) and extracted with ethyl acetate (75 mL×2). The combined ethyl acetate layers were washed with water (two times), dried over anhydrous Na₂SO₄, concentrated and dried to afford a sticky compound (4.34 g, 96% yield).

### Step-2

### Preparation of 2-[4-hydroxyphenyl]-3-(3-methyl-4-fluorophenyl)acrylic acid

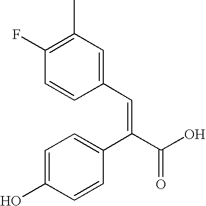

A solution of sodium hydroxide (1.65 g, 41.4 mmol) in water (1.5 mL) was added under stirring to a solution of 2-[4-(acetyloxy)phenyl]-3-(3-methyl-4-fluorophenyl)acrylic acid (4.34 g, 13:8 mmol) in methanol (10 mL) and the whole cooled to 10° C. Then the reaction mixture was stirred for 1 hour at a temperature of 30° C. Subsequently the solvent was removed by evaporation; then water (200 mL) was added and the combined layers were extracted with ethyl acetate (75 mL×2). pH of the aqueous layer was adjusted to 3 using dilute HCl (1:1), the solid obtained was filtered and dried to afford the title compound (1.2 g, 32% yield).

### Step-3

### Preparation of 2-[4-hydroxyphenyl]-3-(3-methyl-4-fluorophenyl)acrylic acid methyl ester

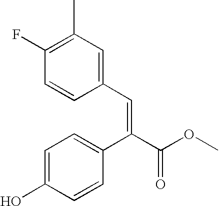

2-[4-hydroxyphenyl]-3-(3-methyl-4-fluorophenyl)acrylic acid (1.2 g, 4.4 mmol) was dissolved in methanol (10 mL) and cooled to 5° C. Then concentrated H₂SO₄ (1 mL) was added under stirring and the reaction mixture heated at 75° C. for 2 hours. Subsequently the solvent was removed by evaporation and the obtained sticky compound was dissolved in ethyl acetate (150 mL). The ethyl acetate layer was washed with water (50 mL×2), dried over anhydrous Na₂SO₄ and the solvent was evaporated to dryness to furnish the title compound (1.03 g, 81% yield).

### Step-4

### Preparation of 2-[4-(oxiran-2-yl-methoxy)phenyl]-3-(3-methyl-4-fluorophenyl)acrylic acid methyl ester

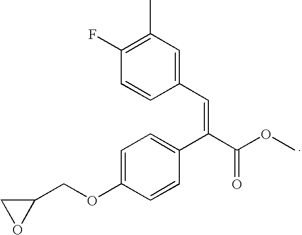

A suspension of 2-[4-hydroxyphenyl]-3-(3-methyl-4-fluorophenyl)acrylic acid methyl ester (1.03 g, 3.6 mmol), epichlorohydrin (0.66 mL, 7.2 mmol) and potassium carbonate (1.49 g, 10 mmol), dissolved in IPA (10 mL) was heated to 80° C. for 4 hours. The solvent was removed by evaporation and the obtained sticky compound was dissolved in ethyl acetate (150 mL). The ethyl acetate layer was washed with water (25 mL×3), dried over anhydrous Na₂SO₄, concentrated and dried to afford the crude compound (0.79 g, 65.5% yield).

### Step-5

### Preparation of 2-{4-[3-(2,4-dimethoxybenzylamino)-2-hydroxypropoxy]phenyl}-3-(4-fluoro-3-methylphenyl)acrylic acid methyl ester

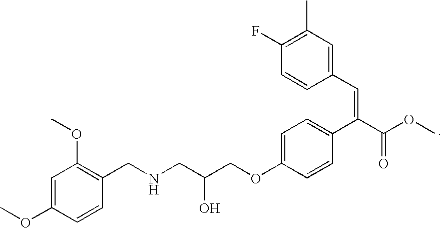

A mixture of methyl 2-[4-(oxiran-2-yl-methoxy)phenyl]3-(3-methyl-4-fluorophenyl)acrylic acid methyl ester (0.27 g, 0.77 mmol) and 2,4-dimethoxybenzylamine (0.23 mL, 1.5 mmol), dissolved in IPA (5 mL) and ethyl acetate (5 mL), was refluxed for 5 hours. Subsequently the solvent was evaporated and the crude compound was purified by flash chromatography. The desired product was eluted in 2.5% methanol in dichloromethane and the solvent was stripped off to furnish the title compound (0.06 g, 14% yield). ¹H NMR (DMSO-d₆) δ (ppm): 2.07 (3H, s, —CH₃), 2.56-2.62 (2H, m, —CH₂), 3.60-3.61 (2H, d, —CH₂), 3.69 (3H, s, —OCH₃), 3.73 (3H, s, —OCH₃), 3.75 (3H, s, —OCH₃), 3.88-3.90 (2H, d, —CH₂), 3.97-3.99 (1H, m, —CH), 4.99 (1H, s, —OH), 6.44-6.46 (1H, m, Ar—H), 6.52-6.53 (1H, d, Ar—H), 6.86-7.00 (4H, m, Ar—H), 7.06-7.11 (3H, m, Ar—H), 7.15-7.17 (1H, d, Ar—H), 7.69 (1H, s, ═CH). MS m/z: 510.5 (M+1).

The following compounds are prepared according to the procedure given in Example 1

| Ex. No | Structure | Analytical Data |
|---|---|---|
| 2 | 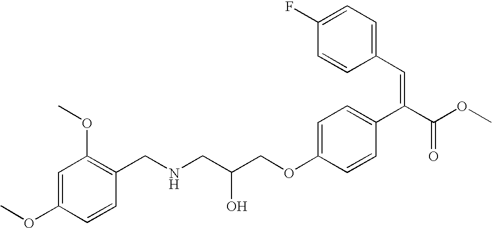 | ¹H NMR (DMSO-d₆) δ (ppm): 2.55-2.66 (2H, m, —CH₂), 3.60-3.61 (2H, d, —CH₂), 3.70 (3H, s, —OCH₃), 3.73 (3H, s, —OCH₃), 3.75 (3H, s, —OCH₃), 3.87-3.89 (2H, d, —CH₂), 3.95-3.98 (1H, m, —CH), 4.99 (1H, s, —OH), 6.44-6.46 (1H, t, Ar—H), 6.52-6.53 (1H, d, Ar—H), 6.93-6.95 (2H, d, Ar—H), 7.06-7.10 (4H, m, Ar—H), 7.13-7.17 (3H, m, Ar—H), 7.74 (1H, s, ═CH). MS m/z: 496.2 (M + 1). |
| 3 | 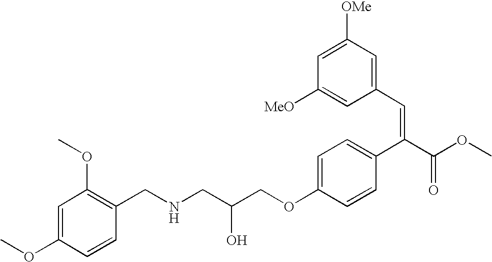 | ¹H NMR (CDCl₃) δ (ppm): 2.9-3.01 (2H, m, —CH₂), 3.54 (6H, s, —OCH₃), 3.63 (3H, s, —OCH₃), 3.70 (3H, s, —OCH₃), 3.78 (3H, s, —OCH₃), 3.96-3.98 (2H, d, —CH₂), 4.04-4.05 (2H, d, —CH₂), 4.15 (1H, m, —CH), 5.76 (1H, s, —OH), 6.28-6.29 (2H, d, Ar—H), 6.39 (1H, s, Ar—H), 6.55-6.58 (1H, m, Ar—H), 6.63- 6.64 (1H, d, Ar—H), 6.95-6.97 (2H, d, Ar— H), 7.10-7.12 (2H, d, Ar—H), 7.31-7.33 (1H, d, Ar—H), 7.68 (1H, s, ═CH). MS m/z: 538.2 (M + 1). |
| 4 | 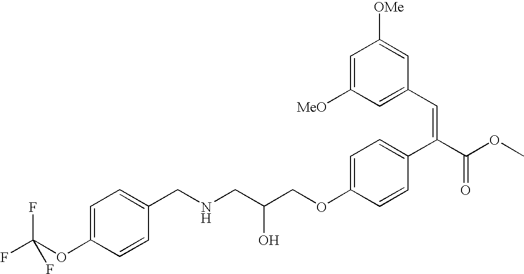 | ¹H NMR (DMSO-d₆) δ (ppm): 2.53-2.66 (2H, m, —CH₂), 3.53 (6H, s, —OCH₃), 3.70 (3H, s, —OCH₃), 3.78 (2H, s, —CH₂), 3.90- 3.91 (2H, d, —CH₂), 4.00-4.01 (1H, m, —CH), 5.10 (1H, s, —OH), 6.27-6.28 (2H, d, Ar—H), 6.38-6.39 (1H, t, Ar—H), 6.94-6.97 (2H, d, Ar—H), 7.08-7.10 (2H, d, Ar—H), 7.28-7.30 (2H, d, Ar—H), 7.45-7.47 (2H, d, Ar—H), 7.67 (1H, s, ═CH). MS m/z: 562.2 (M + 1). |
| 5 | 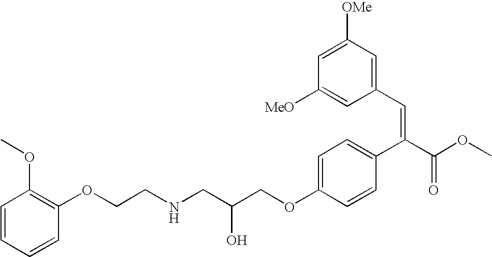 | ¹H NMR (DMSO-d₆) δ (ppm): 2.51-2.52 (2H, m, —CH₂), 2.89-2.92 (2H, t, —CH₂), 3.53 (6H, s, —OCH₃), 3.70 (3H, s, —OCH₃), 3.73 (3H, s, —OCH₃), 3.89-3.91 (2H, d, —CH₂), 3.99-4.02 (3H, m, —CH₂ & —CH), 5.20 (1H, s, —OH), 6.27-6.28 (2H, d, Ar—H), 6.38-6.39 (1H, d, Ar—H), 6.87-6.89 (2H, m, Ar—H), 6.94-6.98 (4H, m, Ar—H), 7.08-7.10 (2H, d, Ar—H), 7.67 (1H, s, ═CH). MS m/z: 538.2 (M + 1). |
| 6 | 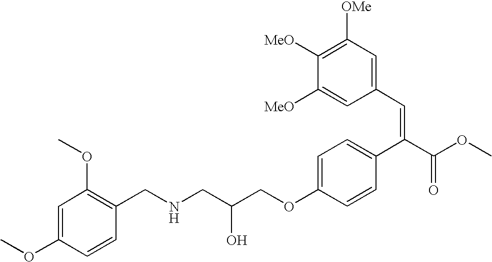 | ¹H NMR (DMSO-d₆) δ (ppm): 2.51-2.67 (2H, m, —CH₂), 3.47 (6H, s, —OCH₃), 3.61 (3H, s, —OCH₃), 3.68-3.70 (2H, d, —CH₂) 3.70 (3H, s, —OCH₃), 3.74 (6H, s, —OCH₃), 3.89-3.98 (3H, m, —CH₂ & —CH), 5.10 (1H, s, —OH), 6.44 (2H, s, Ar—H), 6.46-6.47 (1H, d, Ar—H), 6.48-6.49 (1H, d, Ar—H), 6.99- 7.01 (2H, d, Ar—H), 7.11-7.13 (2H, d, Ar— H), 7.18-7.20 (1H, d, Ar—H), 7.68 (1H, s, ═CH). MS m/z: 568.2 (M + 1). |
| 7 | 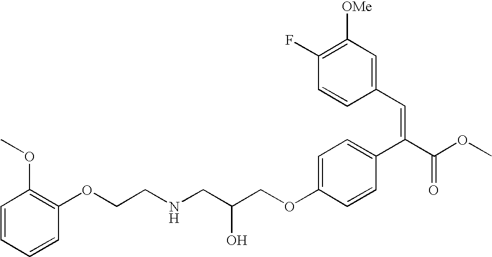 | ¹H NMR (DMSO-d₆) δ (ppm): 2.51-2.54 (2H, m, —CH₂), 2.88-2.91 (2H, t, —CH₂), 3.51 (3H, s, —OCH₃), 3.70 (3H, s, —OCH₃), 3.74 (3H, s, —OCH₃), 3.89-3.91 (2H, t, —CH₂), 3.98-4.02 (3H, m, —CH₂ & —CH), 5.05 (1H, s, —OH), 6.80-6.88 (4H, m, Ar—H), 6.94-6.99 (4H, m, Ar—H), 7.09-7.11 (3H, d, Ar—H), 7.73 (1H, s, ═CH). MS m/z: 526.2 (M + 1). |
| 8 | 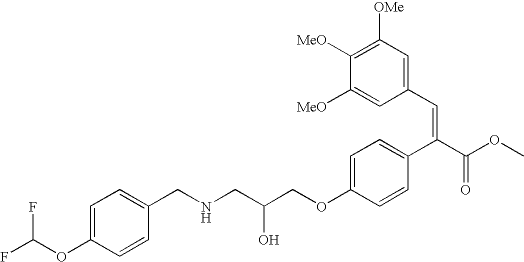 | ¹H NMR (DMSO-d₆) δ (ppm): 2.54-2.67 (2H, m, —CH₂), 3.47 (6H, s, —OCH₃), 3.61 (3H, s, —OCH₃), 3.68 (3H, s, —OCH₃), 3.70- 3.77 (2H, d, —CH₂), 3.89-3.90 (2H, d, —CH₂), 3.99-4.00 (1H, m, —CH), 5.03 (1H, s, —OH), 6.44 (2H, s, Ar—H), 6.98-7.01 (2H, d, Ar—H), 7.10-7.12 (4H, d, Ar—H), 7.19 (1H, s, —CH), 7.37-7.39 (2H, d, Ar—H), 7.70 (1H, s, ═CH). MS m/z: 574.2 (M + 1). |
| 9 | 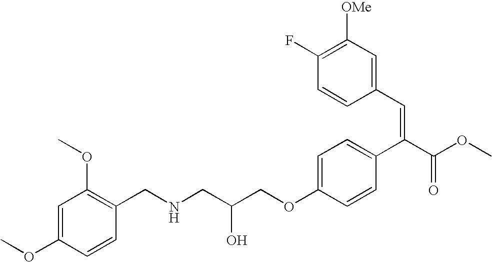 | ¹H NMR (DMSO-d₆) δ (ppm): 2.54-2.61 (2H, m, —CH₂), 3.50 (3H, s, —OCH₃), 3.59- 3.61 (2H, d, —CH₂), 3.71 (3H, s, —OCH₃), 3.73 (3H, s, —OCH₃), 3.75 (3H, s, —OCH₃), 3.88-3.89 (2H, d, —CH₂), 3.98-4.02 (1H, m, —CH), 5.05 (1H, s, —OH), 6.44-6.46 (1H, m, Ar—H), 6.52 (1H, s, Ar—H), 6.80-6.82 (1H, d, Ar—H), 6.97-6.99 (1H, d, Ar—H), 7.07- 7.12 (4H, m, Ar—H), 7.17-7.19 (2H, d, Ar— H), 7.73 (1H, s, ═CH). MS m/z: 526.2 (M + 1). |
| 10 | 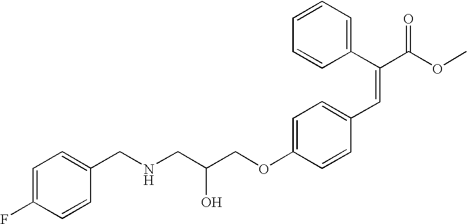 | ¹H NMR (DMSO-d₆) δ (ppm): 2.55-2.59 (2H, m, —CH₂), 3.66-3.68 (5H, m, —CH₂ & —OCH₃), 3.81-3.83 (2H, d, —CH₂), 3.91-3.96 (1H, m, —CH), 4.96 (1H, s, —OH), 6.73-6.75 (2H, d, Ar—H), 6.97-6.99 (2H, d, Ar—H), 7.07-7.11 (2H, m, Ar—H), 7.17-7.19 (2H, d, Ar—H), 7.30-7.32 (2H, d, Ar—H), 7.33-7.43 (3H, m, Ar—H), 7.75 (1H, s, ═CH). MS m/z: 436.1 (M + 1). |
| 11 | 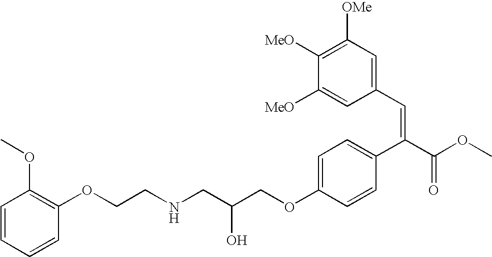 | ¹H NMR (DMSO-d₆) δ (ppm): 2.60-2.74 (2H, m, —CH₂), 2.89-2.91 (2H, t, —CH₂), 3.48 (6H, s, —OCH₃), 3.61 (3H, s, —OCH₃), 3.70 (3H, s, —OCH₃), 3.74 (3H, s, —OCH₃), 3.90-3.93 (2H, t, —CH₂), 3.97-4.02 (3H, m, —CH₂ & —CH), 5.06 (1H, s, —OH), 6.44 (2H, s, Ar—H), 6.86-6.89 (2H, m, Ar—H), 6.94-7.02 (4H, m, Ar—H), 7.10-7.12 (2H, d, Ar—H), 7.70 (1H, s, ═CH). MS m/z: 568.2 (M + 1). |
| 12 | 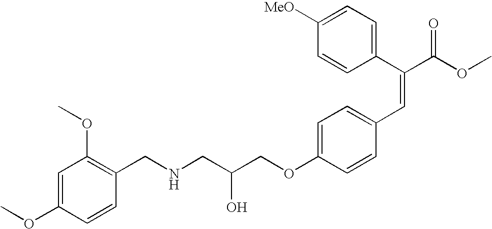 | 1H NMR (DMSO-d₆) δ (ppm): 2.54-2.67 (2H, m, —CH₂), 3.53-3.61 (2H, d, —CH₂), 3.68 (3H, s, —OCH₃), 3.73 (6H, s, —OCH₃), 3.79 (3H, s, —OCH₃), 3.81-3.83 (2H, d, —CH₂), 3.89-3.92 (1H, m, —CH), 4.95 (1H, s, —OH), 6.42-6.44 (1H, d, Ar—H), 6.51 (1H, s, Ar—H), 6.76-6.78 (2H, d, Ar—H), 6.95-6.97 (2H, d, Ar—H), 7.01-7.03 (2H, d, Ar—H), 7.08-7.14 (3H, m, Ar—H), 7.70 (1H, s, ═CH). MS m/z: 508.2 (M + 1). |
| 13 | 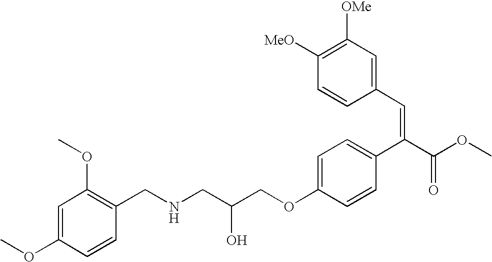 | ¹H NMR (DMSO-d₆) δ (ppm): 2.55-2.62 (2H, m, —CH₂), 3.37 (3H, s, —OCH₃), 3.61- 3.62 (2H, d, —CH₂), 3.69 (3H, s, —OCH₃), 3.71 (3H, s, —OCH₃), 3.74 (3H, s, —OCH₃), 3.76 (3H, s, —OCH₃), 3.88-3.90 (2H, d, —CH₂), 3.97-3.99 (1H, m, —CH), 4.99 (1H, s, —OH), 6.45-6.47 (1H, d, Ar—H), 6.53-6.56 (2H, d, Ar—H), 6.79-6.85 (2H, m, Ar—H), 6.97-6.99 (2H, d, Ar—H), 7.09-7.11 (2H, d, Ar—H), 7.16-7.18 (1H, d, Ar—H), 7.70 (1H, s, ═CH). MS m/z: 538.2 (M + 1). |
| 14 | 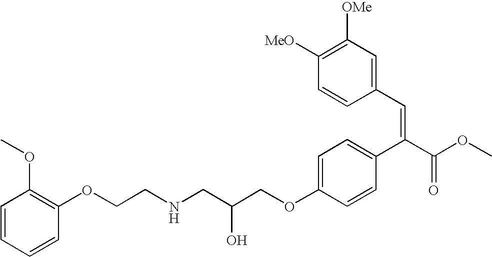 | ¹H NMR (DMSO-d₆) δ (ppm): 2.68-2.77 (2H, m, —CH₂), 2.89-2.92 (2H, t, —CH₂), 3.37 (3H, s, —OCH₃), 3.69 (3H, s, —OCH₃), 3.71 (3H, s, —OCH₃), 3.74 (3H, s, —OCH₃), 3.90-3.92 (2H, d, —CH₂), 3.98-4.03 (3H, m, —CH₂ & —CH), 5.08 (1H, s, —OH), 6.56 (1H, s, Ar—H), 6.81-6.89 (4H, m, Ar—H), 6.95- 7.01 (4H, m, Ar—H), 7.09-7.11 (2H, d, Ar— H), 7.69 (1H, s, ═CH). MS m/z: 538.4 (M + 1). |
| 15 | 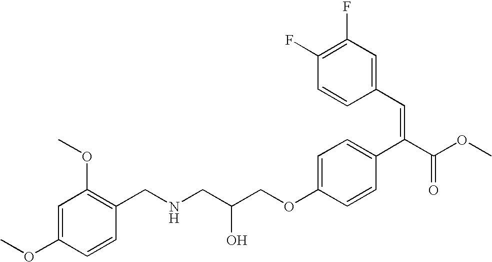 | ¹H NMR (DMSO-d₆) δ (ppm): 2.55-2.66 (2H, m, —CH₂), 3.59-3.61 (2H, d, —CH₂), 3.71 (3H, s, —OCH₃), 3.73 (3H, s, —OCH₃), 3.75 (3H, s, —OCH₃), 3.90 (2H, d, —CH₂), 3.98 (1H, m, —CH), 5.02 (1H, s, —OH), 6.44- 6.46 (1H, d, Ar—H), 6.52 (1H, s, Ar—H), 6.85-6.87 (1H, d, Ar—H), 6.95-6.97 (2H, d, Ar—H), 7.08-7.10 (2H, d, Ar—H), 7.16-7.17 (2H, d, Ar—H), 7.29-7.36 (1H, m, Ar—H) 7.71 (1H, s, ═CH).MS m/z: 514.2 (M + 1). |
| 16 | 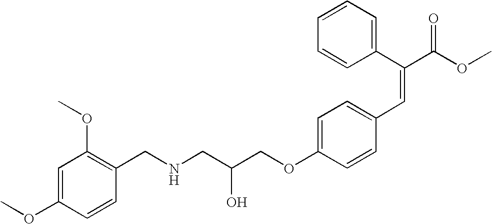 | ¹H NMR (DMSO-d₆) δ (ppm): 2.54-2.55 (2H, m, —CH₂), 3.56-3.57 (2H, d, —CH₂), 3.69 (3H, s, —OCH₃), 3.73 (6H, s, —OCH₃), 3.81-3.82 (2H, d, —CH₂), 3.91 (1H, m, —CH), 4.98 (1H, s, —OH), 6.44 (1H, d, Ar—H), 6.50- 6.51 (1H, d, Ar—H), 6.74-6.76 (2H, d, Ar— H), 6.97-6.99 (2H, d, Ar—H), 7.11-7.13 (3H, d, Ar—H), 7.18-7.19 (3H, d, Ar—H), 7.75 (1H, s, ═CH). MS m/z: 478.2 (M + 1). |
| 17 | 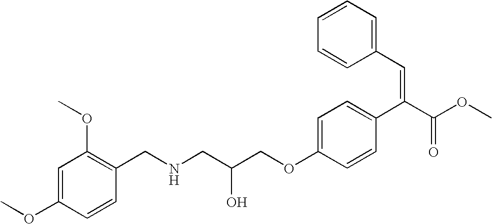 | ¹H NMR (DMSO-d₆) δ (ppm): 2.54-2.65 (2H, m, —CH₂), 3.61 (2H, s, —CH₂), 3.71- 3.75 (9H, s, —OCH₃), 3.88-3.89 (2H, d, —CH₂), 3.96-3.98 (1H, m, —CH), 4.99 (1H, s, —OH), 6.44-6.46 (1H, t, Ar—H), 6.52 (1H, s, Ar—H), 6.92-6.95 (2H, t, Ar—H), 7.07-7.10 (4H, m, Ar—H), 7.16-7.23 (4H, m, Ar—H), 7.74 (1H, s, ═CH). MS m/z: 478.5 (M + 1). |
| 18 | 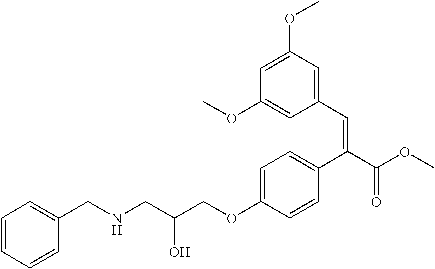 | ¹H NMR (DMSO-d₆) δ (ppm): 2.56-2.67 (2H, m, —CH₂), 3.53 (6H, s, —OCH₃), 3.70 (3H, s, —OCH₃), 3.73-3.76 (2H, d, —CH₂), 3.91-4.01 (3H, m, —CH & —CH₂), 5.01 (1H, s, —OH), 6.28 (2H, s, Ar—H), 6.38 (1H, s, Ar—4H), 6.95-6.97 (2H, d, Ar—H), 7.08- 75.10 (2H, d, Ar—H), 7.22-7.23 (1H, d, Ar— H), 7.28-7.32 (4H, m, Ar—H), 7.67 (1H, s, ═CH). MS m/z: 478.2 (M + 1). |
| 19 | 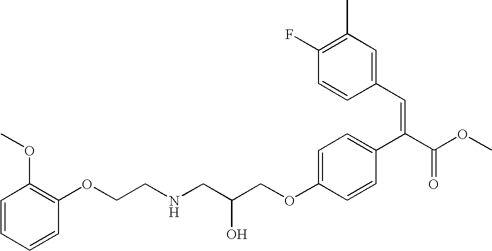 | ¹H NMR (DMSO-d₆) δ (ppm): 2.08 (3H, s, —CH₃), 2.68-2.75 (2H, m, —CH₂), 2.89-2.92 (2H, t, CH₂), 3.70 (3H, s, —OCH₃), 3.73 (3H, s, —OCH₃), 3.90-3.91 (2H, d, —CH₂) 3.98-4.02 (3H, m, —CH₂ & —CH), 5.08 (1H, s, —OH), 6.84-6.89 (3H, m, Ar—H), 6.94- 6.98 (5H, m, Ar—H), 7.00-7.10 (3H, m, Ar— H), 7.69 (1H, s, ═CH). MS m/z: 510.2 (M + 1). |
| 20 | 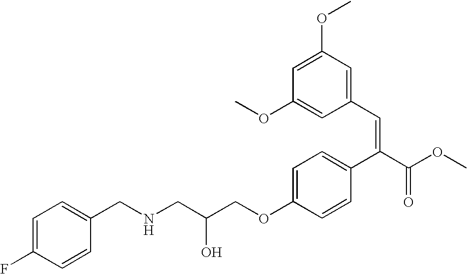 | ¹H NMR (DMSO-d₆) δ (ppm): 2.56-2.65 (2H, m, —CH₂), 3.53 (6H, s, —OCH₃), 3.70 (5H, m, —OCH₃ & —CH₂), 3.89-3.90 (2H, d, —CH₂), 3.99 (1H, m, —CH), 5.01 (1H, s, —OH), 6.28 (2H, s, Ar—H), 6.38-6.39 (1H, d, Ar—H), 6.95-6.97 (2H, d, Ar—H), 7.08-7.13 (4H, m, Ar—H), 7.34-7.37 (2H, m, Ar—H), 7.67 (1H, s, ═CH). MS m/z: 496.2 (M + 1). |
| 21 | 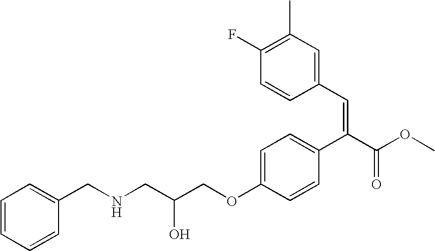 | ¹H NMR (DMSO-d₆) δ (ppm): 2.07 (3H, s, —CH₃), 2.56-2.67 (2H, m, —CH₂), 3.70 (3H, s, —OCH₃), 3.73 (2H, d, —CH₂), 3.89-3.91 (2H, d, —CH₂), 4.00-4.02 (1H, m, —CH), 5.01 (1H, s, —OH), 6.86-6.88 (1H, m, Ar—H), 6.94-6.99 (3H, m, Ar—H), 7.06-7.10 (3H, m, Ar—H), 7.21-7.23 (1H, m, Ar—H), 7.28-7.33 (4H, m, Ar—H), 7.69 (1H, s, ═CH). MS m/z: 450.2 (M + 1). |
| 22 | 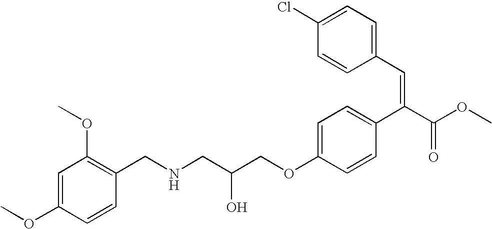 | ¹H NMR (DMSO-d₆) δ (ppm): 2.52-2.60 (2H, m, —CH₂), 3.61-3.62 (2H, d, —CH₂), 3.71-3.75 (9H, s, —OCH₃), 3.88-3.89 (2H, d, —CH₂), 3.96-3.98 (1H, m, —CH), 4.99 (1H, s, —OH), 6.44-6.47 (1H, m, Ar—H), 6.52-6.53 (1H, d, Ar—H), 6.93-6.95 (2H, t, Ar—H), 7.06-7.12 (3H, m, Ar—H), 7.15-7.17 (2H, d, Ar—H), 7.28-7.31 (2H, t, Ar—H), 7.72 (1H, s, ═CH). MS m/z: 512.2 (M + 1). |
| 23 | 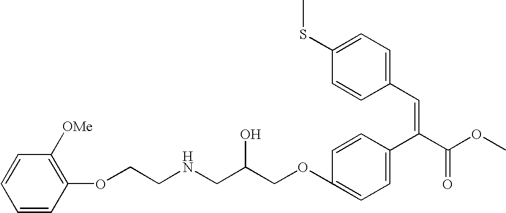 | ¹H NMR (DMSO-d₆) δ (ppm): 2.41 (3H, s, —SCH₃), 2.67-2.77 (2H, m, —CH₂), 2.91 (2H, t, —CH₂), 3.69 (3H, s, —OCH₃), 3.74 (3H, s, —OCH₃), 3.90-3.91 (2H, d, —CH₂), 4.00-4.01 (3H, m, —CH₂ & —CH), 5.08 (1H, s, —OH), 6.86-6.90 (2H, m, Ar—H), 6.94-6.96 (4H, m, Ar—H), 7.01-7.03 (2H, d, Ar—H), 7.07-7.09 (4H, m, Ar—H), 7.69 (1H, s, ═CH). MS m/z: 524.2 (M + 1). |
| 24 | 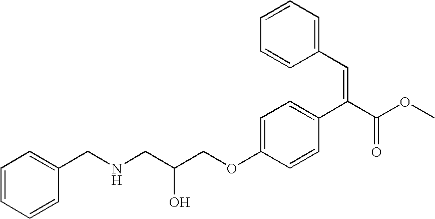 | ¹H NMR (DMSO-d₆) δ (ppm): 2.56-2.67 (2H, m, —CH₂), 3.70-3.73 (5H, m, —CH₂ & —OCH₃), 3.89-3.91 (2H, d, —CH₂), 3.99-4.01 (1H, m, —CH), 5.03 (1H, s, —OH), 6.92-6.94 (2H, d, Ar—H), 7.07-7.11 (4H, m, Ar—H), 7.20-7.23 (4H, m, Ar—H), 7.28-7.32 (4H, m, Ar—H), 7.74 (1H, s, ═CH). MS m/z: 418.2 (M + 1). |
| 25 | 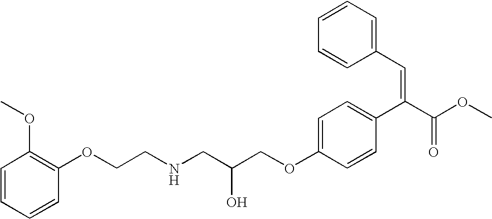 | ¹H NMR (DMSO-d₆) δ (ppm): 2.67-2.75 (2H, m, —CH₂), 2.89-2.91 (2H, t, —CH₂), 3.71 (3H, s, —OCH₃), 3.73 (3H, s, —OCH₃), 3.90-3.91 (2H, d, —CH₂), 3.96-4.02 (3H, m, —CH & —CH₂), 5.07 (1H, s, —OH), 6.86-6.89 (2H, m, Ar—H), 6.93-6.98 (4H, m, Ar—H), 7.07-7.11 (4H, m, Ar—H), 7.22-7.23 (3H, d, Ar—H), 7.74 (1H, s, ═CH). MS m/z: 478.2 (M + 1). |
| 26 | 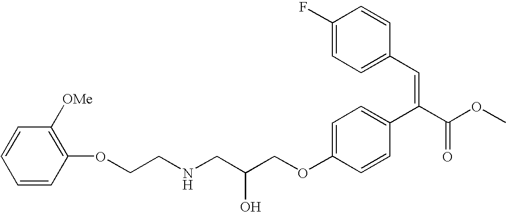 | ¹H NMR (DMSO-d₆) δ (ppm): 2.65-2.77 (2H, m, —CH₂), 2.89-2.90 (2H, t, —CH₂), 3.70 (3H, s, —OCH₃), 3.74 (3H, s, —OCH₃), 3.90-3.91 (2H, d, —CH₂), 3.98-4.01 (3H, m, —CH & —CH₂), 5.09 (1H, s, —OH), 6.89-6.94 (2H, m, Ar—H), 6.96-6.98 (4H, m, Ar—H), 7.03-7.08 (4H, m, Ar—H), 7.12-7.16 (2H, d, Ar—H), 7.74 (1H, s, ═CH). MS m/z: 496.2 (M + 1). |
| 27 | 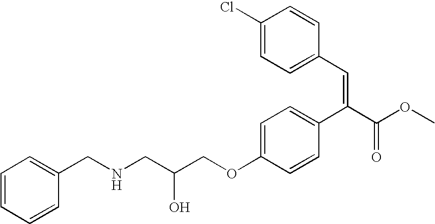 | ¹H NMR (DMSO-d₆) δ (ppm): 2.56-2.67 (2H, m, —CH₂), 3.70 (3H, s, —OCH₃), 3.73 (2H, d, —CH₂), 3.87-3.91 (2H, m, —CH₂), 3.99-4.01 (1H, m, —CH), 5.03 (1H, s, —OH), 6.93-6.95 (2H, d, Ar—H), 7.07-7.11 (4H, m, Ar—H), 7.20-7.23 (1H, m, Ar—H), 7.23-7.34 (6H, m, Ar—H), 7.72 (1H, s, ═CH). MS m/z: 452.1 (M + 1). |
| 28 | 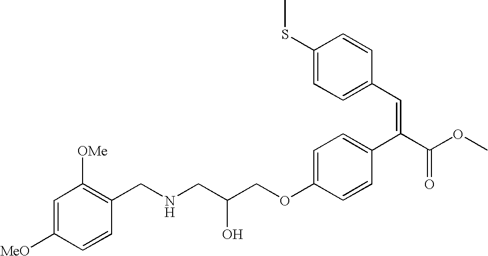 | ¹H NMR (DMSO-d₆) δ (ppm): 2.42 (3H, s, —SCH₃), 2.57-2.65 (2H, m, —CH₂), 3.58-3.61 (2H, d, —CH₂), 3.69-3.77 (9H, s, —OCH₃), 3.90-3.99 (1H, m, —CH₂ & —CH), 5.03 (1H, s, —OH), 6.44-6.46 (1H, d, Ar—H), 6.52 (1H, s, Ar—H), 6.94-6.96 (2H, d, Ar—H), 7.01- 7.03 (2H, d, Ar—H), 7.07-7.09 (4H, m, Ar— H), 7.16-7.18 (1H, d, Ar—H), 7.70 (1H, s, ═CH). MS m/z: 524.2 (M + 1). |
| 29 | 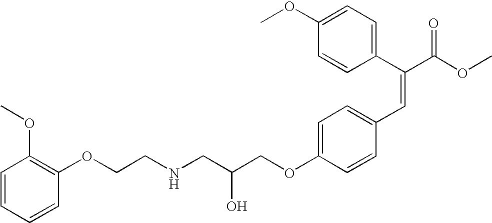 | ¹H NMR (DMSO-d₆) δ (ppm): 2.61-2.67 (2H, m, —CH₂), 2.84-2.87 (2H, t, —CH₂), 3.68-3.74 (9H, s, —OCH₃), 3.82-3.84 (2H, d, —CH₂), 3.89-3.91 (1H, m, —CH), 3.96-3.99 (2H, t, —CH₂), 5.03 (1H, s, —OH), 6.76-6.78 (2H, d, Ar—H), 6.84-6.90 (2H, m, Ar—H), 6.93-6.97 (4H, m, Ar—H), 7.01-7.03 (2H, d, Ar—H), 7.08-7.10 (2H, d, Ar—H), 7.70 (1H, s, ═CH). MS m/z: 508.23 (M + 1). |
| 30 | 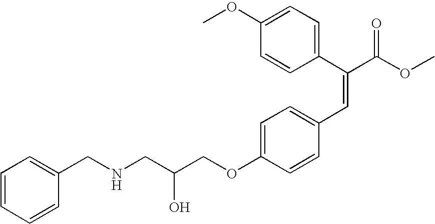 | ¹H NMR (DMSO-d₆) δ (ppm): 2.57-2.60 (2H, m, —CH₂), 3.68 (5H, m, —OCH₃ & —CH₂), 3.79 (3H, s, —OCH₃), 3.84 (2H, d, —CH₂), 3.94 (1H, m, —CH), 4.97 (1H, s, —OH), 6.76-6.78 (2H, d, Ar—H), 6.95-6.97 (2H, d, Ar—H), 7.01-7.03 (2H, d, Ar—H), 7.08-7.10 (2H, d, Ar—H), 7.20 (1H, m, Ar— H), 7.29 (4H, m, Ar—H), 7.70 (1H, s, ═CH). MS m/z: 448.2 (M + 1). |
| 31 | 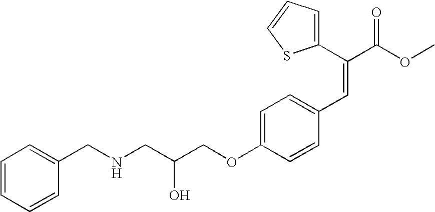 | ¹H NMR (DMSO-d₆) δ (ppm): 2.57-2.60 (2H, m, —CH₂), 3.69 (2H, d, —CH₂), 3.72 (3H, s, —OCH₃), 3.85-3.86 (2H, d, —CH₂), 3.97 (1H, m, —CH), 4.99 (1H, s, —OH), 6.81- 6.83 (2H, d, Ar—H), 6.96 (1H, m, Ar—H), 7.10-7.11 (3H, d, Ar—H), 7.21 (1H, m, Ar— H), 7.28-7.30 (4H, m, Ar—H), 7.65-7.66 (1H, d, Ar—H), 7.83 (1H, s, ═CH). MS m/z: 424.1 (M + 1). |
| 32 | 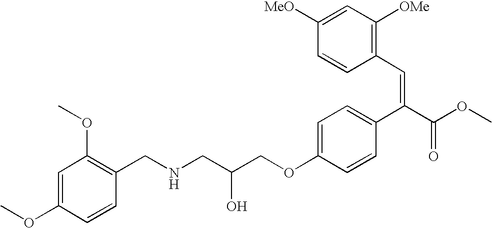 | ¹H NMR (DMSO-d₆) δ (ppm): 2.56-2.64 (2H, m, —CH₂), 3.57-3.61 (2H, d, —CH₂), 3.68-3.79 (12H, s, —OCH₃), 3.84 (3H, s, —OCH₃), 3.88 (2H, m, —CH₂), 3.94-3.97 (1H, m, —CH), 5.00 (1H, s, —OH), 6.24-6.26 (1H, d, Ar—H), 6.44-6.46 (1H, d, Ar—H), 6.52 (1H, s, Ar—H), 6.56-6.58 (2H, d, Ar—H), 6.89-6.91 (2H, d, Ar—H), 7.03-7.05 (2H, d, Ar—H), 7.15-7.17 (1H, d, Ar—H), 7.94 (1H, s, ═CH). MS m/z: 538.2 (M + 1). |
| 33 | 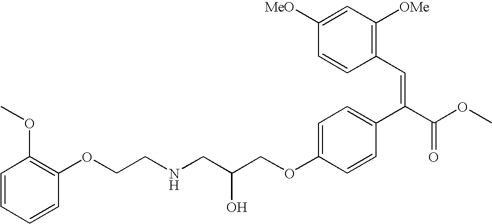 | ¹H NMR (DMSO-d₆) δ (ppm): 2.60-2.70 (2H, m, —CH₂), 2.89-2.91 (2H, t, —CH₃), 3.68-3.73 (9H, s, —OCH₃), 3.85 (3H, s, —OCH₃), 3.883.90 (2H, d, —CH₂), 3.96-4.01 (1H, m, —CH), 4.02 (2H, d, —CH₂), 5.08 (1H, s, —OH), 6.24-6.26 (1H, d, Ar—H), 6.56-6.58 (2H, d, Ar—H), 6.86-6.88 (2H, m, Ar—H), 6.90-6.97 (4H, m, Ar—H), 7.04-7.06 (2H, d, Ar—H), 7.94 (1H, s, ═CH). MS m/z: 538.2 (M + 1). |
| 34 | 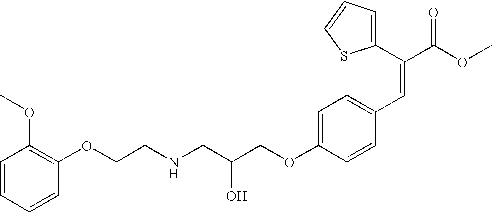 | ¹H NMR (DMSO-d₆) δ (ppm): 2.59-2.71 (2H, m, —CH₂), 2.85-2.86 (2H, t, CH₂), 3.72 (6H, s, —OCH₃), 3.85-3.89 (2H, m, —CH₂), 3.94-3.98 (3H, m, —CH₂ & —CH), 5.05 (1H, s, —OH), 6.81-6.90 (4H, m, Ar—H), 6.94- 6.96 (3H, m, Ar—H), 7.10-7.12 (3H, d, Ar— H), 7.65-7.66 (1H, m, Ar—H), 7.83 (1H, s, ═CH). MS m/z: 484.2 (M + 1). |
| 35 | 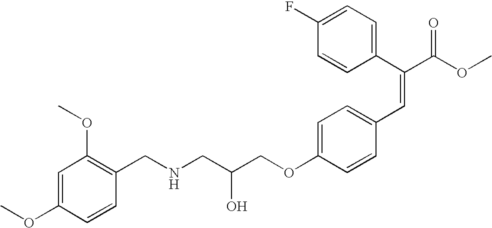 | ¹H NMR (DMSO-d₆) δ (ppm): 2.54-2.55 (2H, m, —CH₂), 3.56-3.57 (2H, d, —CH₂), 3.69 (3H, s, —OCH₃), 3.73 (6H, s, —OCH₃), 3.81-3.83 (2H, d, —CH₂), 3.90-3.93 (1H, m, —CH), 4.96 (1H, s, —OH), 6.41-6.44 (1H, m, Ar—H), 6.50 (1H, s, Ar—H), 6.77-6.79 (2H, d, Ar—H), 6.98-7.01 (2H, d, Ar—H), 7.11- 7.13 (1H, d, Ar—H), 7.22-7.25 (4H, m, Ar— H), 7.76 (1H, s, ═CH). MS m/z: 496.4 (M + 1). |
| 36 | 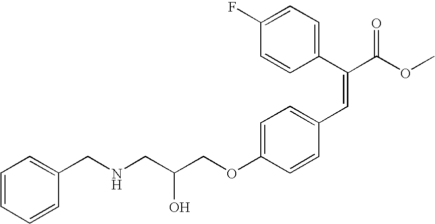 | ¹H NMR (DMSO-d₆) δ (ppm): 2.54-2.58 (2H, m, —CH₂), 3.69 (5H, m, —CH₂ & —OCH₃), 3.83-3.84 (2H, d, —CH₂), 3.94-3.96 (1H, m, —CH), 4.99 (1H, s, —OH), 6.77-6.79 (2H, m, Ar—H), 6.99-7.01 (2H, d, Ar—H), 7.21-7.25 (5H, m, Ar—H), 7.28-7.29 (4H, m, Ar—H), 7.76 (1H, s, ═CH). MS m/z: 436.2 (M + 1). |
| 37 | 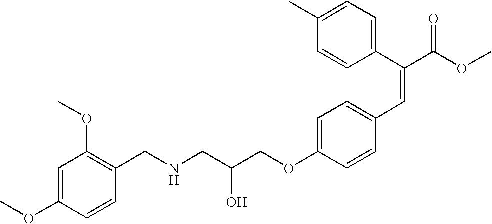 | ¹H NMR (DMSO-d₆) δ (ppm): 2.35 (3H, s, —CH₃), 2.51-2.55 (2H, m, —CH₂), 3.56-3.57 (2H, d, —CH₂), 3.67 (3H, s, —OCH₃), 3.73 (6H, s, —OCH₃), 3.83-3.84 (2H, d, —CH₂), 3.91-3.93 (1H, m, —CH), 4.93 (1H, s, —OH), 6.42-6.44 (1H, m, Ar—H), 6.50 (1H, s, Ar— H), 6.75-6.77 (2H, d, Ar—H), 7.00-7.07 (4H, m, Ar—H), 7.12-7.14 (1H, d, Ar—H), 7.20- 7.22 (2H, m, Ar—H), 7.71 (1H, s, ═CH). MS m/z: 492.5 (M + 1). |
| 38 | 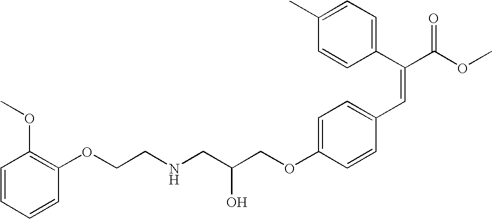 | ¹H NMR (DMSO-d₆) δ (ppm): 2.35 (3H, s, —CH₃), 2.60-2.70 (2H, m, —CH₂), 2.84-2.87 (2H, t, —CH₂), 3.67 (3H, s, —OCH₃), 3.71 (3H, s, —OCH₃), 3.83-3.84 (2H, d, —CH₂), 3.91-3.98 (3H, m, —CH₂ & —CH), 5.03 (1H, s, —OH), 6.75-6.77 (2H, d, Ar—H), 6.85-6.89 (2H, m, Ar—H), 6.93-6.95 (2H, d, Ar—H), 7.00-7.07 (4H, m, Ar—H), 7.21-7.23 (2H, d, Ar—H), 7.71 (1H, s, ═CH). MS m/z: 492.4 (M + 1). |
| 39 | 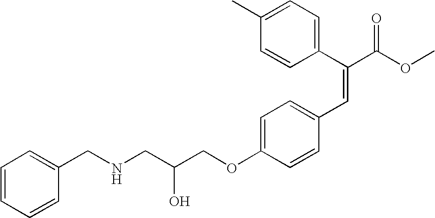 | ¹H NMR (DMSO-d₆) δ (ppm): 2.35 (3H, s, —CH₃), 2.51-2.56 (2H, m, —CH₂), 3.67 (5H, m, —CH₂ & —OCH₃), 3.81-3.83 (2H, d, —CH₂), 3.91-3.93 (1H, m, —CH), 4.98 (1H, s, —OH), 6.74-6.76 (2H, m, Ar—H), 7.00-7.07 (4H, m, Ar—H), 7.20-7.29 (7H, m, Ar—H), 7.71 (1H, s, ═CH). MS m/z: 432.5 (M + 1). |
| 40 | 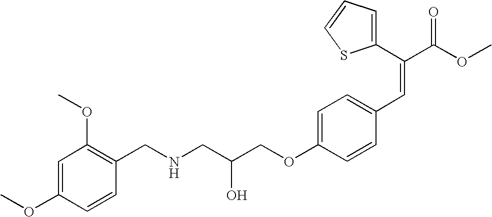 | ¹H NMR (DMSO-d₆) δ (ppm): 2.54-2.57 (2H, m, —CH₂), 3.58 (2H, d, —CH₂), 3.72- 3.75 (9H, s, —OCH₃), 3.84-3.86 (2H, d, —CH₂), 3.94-3.96 (1H, m, —CH), 4.98 (1H, s, —OH), 6.43-6.45 (1H, d, Ar—H), 6.51 (1H, s, Ar—H), 6.81-6.83 (2H, d, Ar—H), 6.97 (1H, s, Ar—H), 7.10-7.15 (4H, m, Ar—H), 7.65- 7.66 (1H, d, Ar—H), 7.83 (1H, s, ═CH). MS m/z: 484.4 (M + 1). |
| 41 | 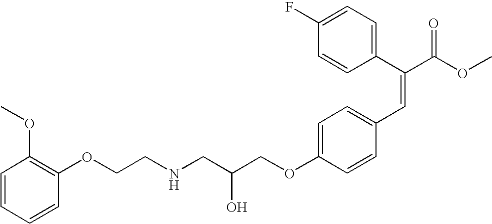 | ¹H NMR (DMSO-d₆) δ (ppm): 2.61-2.68 (2H, m, —CH₂), 2.85-2.87 (2H, t, —CH₂), 3.69-3.72 (6H, s, —OCH₃), 3.83-3.85 (2H, d, —CH₂), 3.92-3.99 (3H, m, —CH & —CH₂), 5.03 (1H, s, —OH), 6.78-6.80 (2H, d, Ar—H), 6.85-6.90 (2H, m, Ar—H), 6.94-6.95 (2H, d, —Ar—H), 6.98-7.00 (2H, d, Ar—H), 7.23-7.25 (4H, m, Ar—H), 7.76 (1H, s, ═CH). MS m/z: 496.10 (M + 1). |
| 42 | 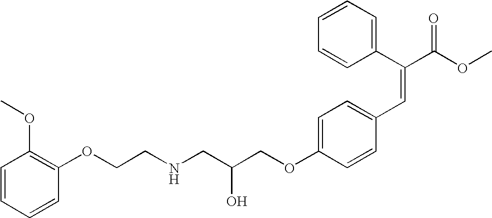 | ¹H NMR (DMSO-d₆) δ (ppm): 2.60-2.66 (2H, m, —CH₂), 2.85 (2H, t, —CH₂), 3.69- 3.71 (6H, s, —OCH₃), 3.82-3.84 (2H, t, —CH₂), 3.91-3.97 (3H, m, —CH₂ & —CH), 5.02 (1H, s, —OH), 6.74-6.76 (2H, m, Ar—H), 6.86-6.88 (2H, m, Ar—H), 6.93-6.99 (4H, m, Ar—H), 7.17-7.19 (2H, d, Ar—H), 7.40-7.42 (3H, m, Ar—H), 7.75 (1H, s, ═CH). MS m/z: 478.2 (M + 1). |
| 43 | 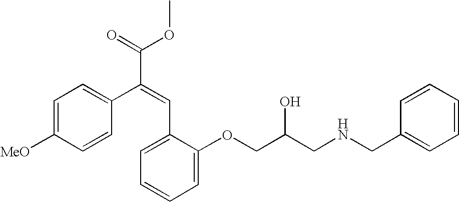 | ¹H NMR (DMSO-d₆) δ (ppm): 2.64-2.66 (2H, m, —CH₂), 3.69 (3H, s, —OCH₃), 3.74- 3.75 (5H, d, —OCH₃ & —CH₂), 3.97-4.00 (2H, d, —CH₂), 4.06 (1H, m, —CH), 5.06 (1H, s, —OH), 6.61-6.63 (2H, m, Ar—H), 6.88- 6.90 (2H, d, Ar—H), 7.02-7.07 (3H, m, Ar— H), 7.19-7.22 (2H, m, Ar—H), 7.27-7.34 (4H, m, Ar—H), 7.98 (1H, s, ═CH). MS m/z: 448.0 (M + 1). |
| 44 | 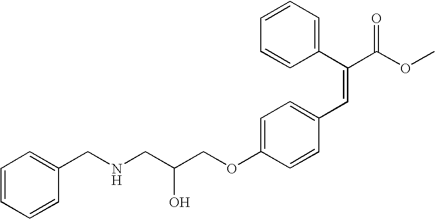 | ¹H NMR (DMSO-d₆) δ (ppm): 2.56-2.60 (2H, m, —CH₂), 3.69 (5H, m, —OCH₃ & —CH₂), 3.82-3.84 (2H, d, —CH₂), 3.94-3.95 (1H, m, —CH), 4.98 (1H, s, —OH), 6.74-6.76 (2H, d, Ar—H), 6.98-7.00 (2H, d, Ar—H), 7.18-7.30 (7H, m, Ar—H), 7.41-7.43 (3H, m, Ar—H), 7.75 (1H, s, ═CH). MS m/z: 418.0 (M + 1). |
| 45 | 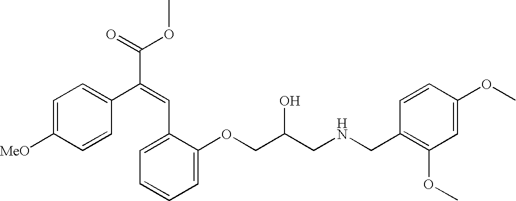 | ¹H NMR (DMSO-d₆) δ (ppm): 2.61-2.68 (2H, m, —CH₂), 3.62-3.63 (2H, d, —CH), 3.70-3.75 (12H, s, —OCH₃), 3.99-4.02 (3H, m, —CH₂ & —CH), 5.03 (1H, s, —OH), 6.43- 6.45 (1H, m, Ar—H), 6.51-6.52 (1H, d, Ar— H), 6.61-6.63 (2H, m, Ar—H), 6.88-6.90 (2H, d, —Ar—H), 7.02-7.07 (3H, m, Ar—H), 7.16-7.18 (2H, m, Ar—H), 7.99 (1H, s, ═CH). MS m/z: 508.0 (M + 1). |
| 46 | 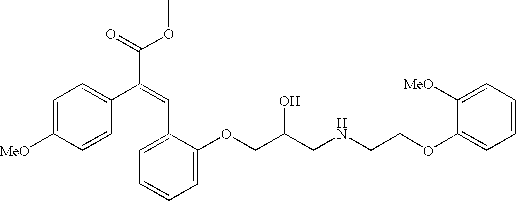 | ¹H NMR (DMSO-d₆) δ (ppm): 2.72-2.83 (2H, m, —CH₂), 2.91-2.93 (2H, t, —CH₂), 3.69 (3H, s, —OCH₃), 3.73-3.75 (6H, s, —OCH₃), 3.96-3.98 (1H, m, —CH), 4.02 (4H, m, —CH₂), 5.11 (1H, s, —OH), 6.62-6.63 (2H, d, Ar—H), 6.85-6.89 (4H, m, Ar—H), 6.94- 6.97 (2H, m, Ar—H), 7.02-7.07 (3H, m, Ar— H), 7.19-7.20 (1H, d, Ar—H), 8.01 (1H, s, ═CH). MS m/z: 508.0 (M + 1). |
| 47 | 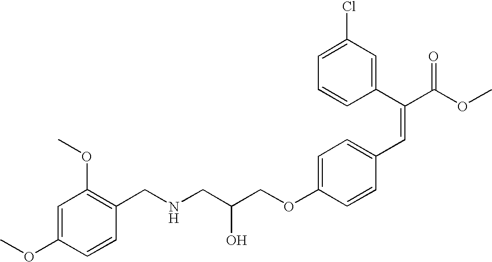 | ¹H NMR (DMSO-d₆) δ (ppm): 2.54-2.57 (2H, m, —CH₂), 3.57-3.58 (2H, d, —CH₂), 3.70 (3H, s, —OCH₃), 3.73 (6H, s, —OCH₃), 3.82-3.84 (2H, d, —CH₂), 3.90-3.94 (1H, m, —CH), 4.96 (1H, s, —OH), 6.42-6.44 (1H, m, Ar—H), 6.50-6.51 (1H, d, Ar—H), 6.79-6.81 (2H, d, Ar—H), 6.99-7.02 (2H, d, Ar—H), 7.12-7.17 (2H, m, Ar—H), 7.29 (1H, s, Ar— H), 7.44-7.45 (2H, d, Ar—H), 7.78 (1H, s, ═CH). MS m/z: 511.9 (M + 1). |
| 48 | 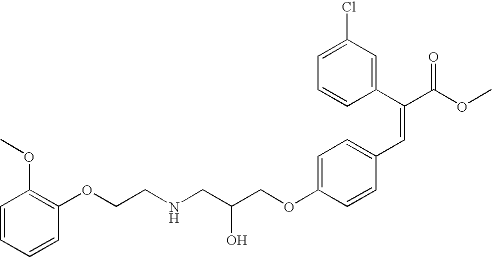 | ¹H NMR (DMSO-d₆) δ (ppm): 2.58-2.70 (2H, m, —CH₂), 2.85-2.87 (2H, t, —CH₂), 3.70 (6H, s, —OCH₃), 3.83-3.87 (2H, t, —CH₂), 3.93-3.99 (3H, m, —CH₂ & —CH), 5.04 (1H, s, —OH), 6.79-6.81 (2H, d, Ar—H), 6.85-6.88 (2H, m, Ar—H), 6.93-6.95 (2H, d, Ar—H), 6.99-7.02 (2H, d, Ar—H), 7.20-7.22 (1H, d, Ar—H), 7.28 (1H, s, Ar—H), 7.44- 7.46 (2H, d, Ar—H), 7.78 (1H, s, ═CH). MS m/z: 511.9 (M + 1). |
| 49 | 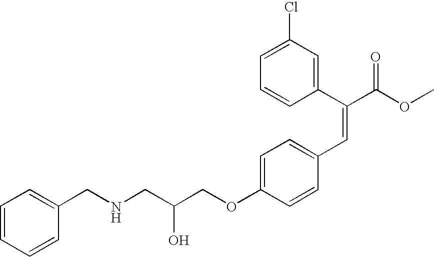 | ¹H NMR (DMSO-d₆) δ (ppm): 2.54-2.61 (2H, m, —CH₂), 3.70 (5H, m, —OCH₃ & —CH₂), 3.83-3.85 (2H, d, —CH₂), 3.93-3.96 (1H, m, —CH), 4.98 (1H, s, —OH), 6.78-6.80 (2H, d, Ar—H), 7.00-7.02 (2H, d, Ar—H), 7.16-7.18 (1H, m, Ar—H), 7.20-7.22 (1H, m, Ar—H), 7.26-7.29 (5H, m, Ar—H), 7.44-7.46 (2H, m, Ar—H), 7.78 (1H, s, ═CH). MS m/z: 452.0 (M + 1). |
| 50 | 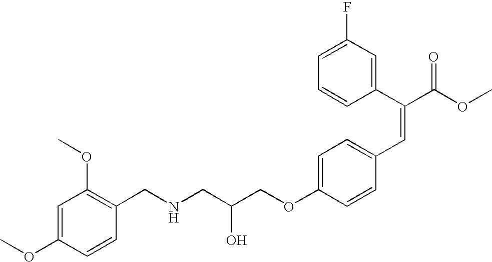 | ¹H NMR (DMSO-d₆) δ (ppm): 2.54-2.57 (2H, m, —CH₂), 3.56-3.57 (2H, d, —CH₂), 3.70-3.75 (9H, s, —OCH₃), 3.82-3.83 (2H, d, —CH₂), 3.92-3.93 (1H, m, —CH), 4.95 (1H, s, —OH), 6.42-6.44 (1H, m, Ar—H), 6.50-6.51 (1H, d, Ar—H), 6.78-6.79 (2H, d, Ar—H), 6.99-7.03 (3H, m, Ar—H), 7.06-7.14 (2H, m, Ar—H), 7.22-7.23 (1H, m, Ar—H), 7.44-7.46 (1H, m, Ar—H), 7.77 (1H, s, ═CH). MS m/z: 496.0 (M + 1). |
| 51 | 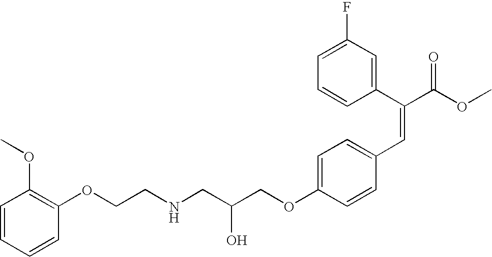 | ¹H NMR (DMSO-d₆) δ (ppm): 2.58-2.67 (2H, m, —CH₂), 2.85-2.87 (2H, t, —CH₂), 3.69-3.72 (6H, s, —OCH₃), 3.83-3.85 (2H, d, —CH₂), 3.92-3.94 (1H, m, —CH), 3.96-3.99 (2H, t, —CH₂), 5.04 (1H, s, —OH), 6.78-6.80 (2H, d, Ar—H), 6.84-6.88 (2H, m, Ar—H), 6.93-6.95 (2H, d, Ar—H), 6.99-7.07 (4H, m, Ar—H), 7.22-7.23 (1H, m, Ar—H), 7.45-7.46 (1H, m, Ar—H), 7.78 (1H, s, ═CH). MS m/z: 496.1 (M + 1). |
| 52 | 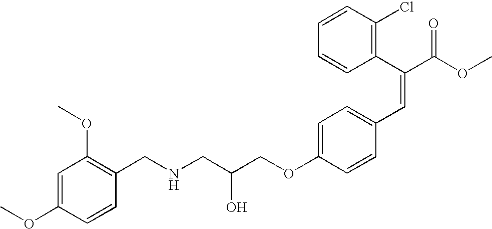 | ¹H NMR (DMSO-d₆) δ (ppm): 2.54-2.57 (2H, m, —CH₂), 3.51-3.53 (2H, d, —CH₂), 3.69 (3H, s, —OCH₃), 3.73 (6H, s, —OCH₃), 3.82-3.83 (2H, d, —CH₂), 3.92-3.93 (1H, m, —CH), 4.98 (1H, s, —OH), 6.42-6.44 (1H, d, Ar—H), 6.50-6.51 (1H, d, Ar—H), 6.78-6.79 (2H, d, Ar—H), 6.96-6.98 (2H, d, Ar—H), 7.11-7.13 (1H, d, Ar—H), 7.23-7.25 (1H, d, Ar—H), 7.37-7.40 (1H, m, Ar—H), 7.43-7.47 (1H, m, Ar—H), 7.58-7.56 (1H, d, Ar—H), 7.83 (1H, s, ═CH). MS m/z: 512.1 (M + 1). |
| 53 | 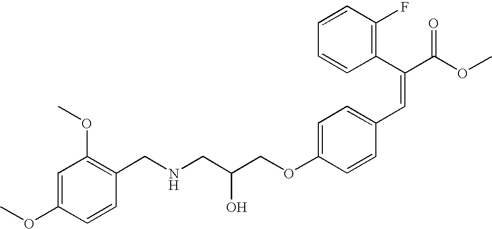 | ¹H NMR (DMSO-d₆) δ (ppm): 2.54-2.57 (2H, m, —CH₂), 3.57-3.58 (2H, d, —CH₂), 3.70 (3H, s, —OCH₃), 3.73 (6H, s, —OCH₃), 3.82-3.85 (2H, t, —CH₂), 3.93-3.94 (1H, m, —CH), 5.02 (1H, s, —OH), 6.42-6.44 (1H, m, Ar—H), 6.50-6.51 (1H, d, Ar—H), 6.78-6.80 (2H, d, Ar—H), 7.03-7.05 (2H, d, Ar—H), 7.12-7.13 (1H, d, Ar—H), 7.23-7.31 (3H, m, Ar—H), 7.47-7.48 (1H, m, Ar—H), 7.86 (1H, s, ═CH). MS m/z: 496.1 (M + 1). |
| 54 | 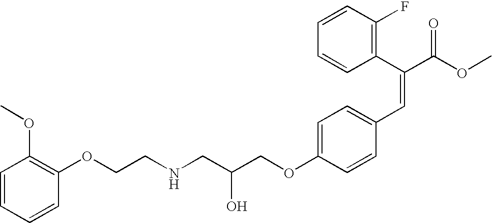 | ¹H NMR (DMSO-d₆) δ (ppm): 2.60-2.68, (2H, m, —CH₂), 2.85-2.87 (2H, t, —CH₂), 3.70-3.71 (6H, s, —OCH₃), 3.84-3.86 (2H, d, —CH₂), 3.93-3.98 (3H, m, —CH & —CH₂), 5.04 (1H, s, —OH), 6.79-6.81 (2H, d, Ar—H), 6.85-6.88 (2H, m, Ar—H), 6.93-6.95 (2H, m, Ar—H), 7.03-7.05 (2H, d, Ar—H), 7.23-7.29 (3H, m, Ar—H), 7.46 (1H, m, Ar—H), 7.87 (1H, s, ═CH). MS m/z: 496.0 (M + 1). |
| 55 | 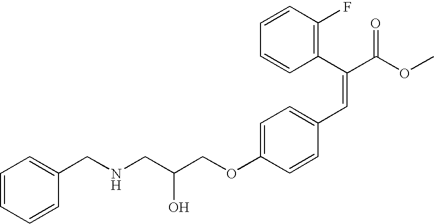 | ¹H NMR (DMSO-d₆) δ (ppm): 2.52-2.60 (2H, m, —CH₂), 3.68-3.72 (5H, t, —OCH₃ & —CH₂), 3.83-3.85 (2H, d, —CH₂), 3.94-3.97 (1H, m, —CH), 4.98 (1H, s, —OH), 6.78-6.80 (2H, d, Ar—H), 7.03-7.05 (2H, d, Ar—H), 7.20-7.31 (8H, m, Ar—H), 7.47-7.49 (1H, m, Ar—H), 7.87 (1H, s, ═CH). MS m/z: 436.1 (M + 1). |
| 56 | 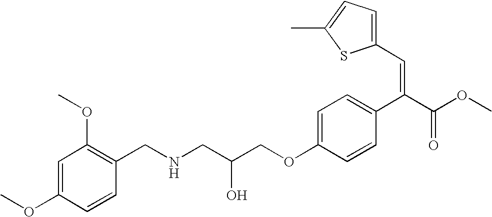 | ¹H NMR (DMSO-d₆) δ (ppm): 2.28 (3H, s, —CH₃), 2.56-2.67 (2H, m, —CH₂), 3.62-3.65 (5H, t, —OCH₃ & —CH₂), 3.73-3.76 (6H, s, —OCH₃), 3.91-3.93 (2H, d, —CH₂), 3.99-4.02 (1H, m, —CH), 5.03 (1H, s, —OH), 6.45-6.47 (1H, d, Ar—H), 6.53 (1H, s, Ar—H), 6.74 (1H, s, Ar—H), 6.99-7.00 (2H, d, Ar—H), 7.08-7.10 (2H, d, Ar—H), 7.16-7.18 (1H, d, Ar—H), 7.28-7.29 (1H, d, Ar—H), 7.93 (1H, s, ═CH). MS m/z: 498.1 (M + 1). |
| 57 | 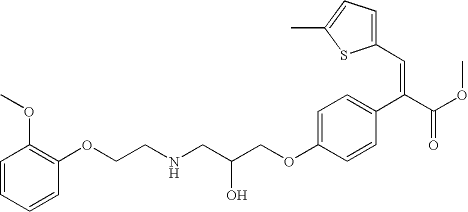 | ¹H NMR (DMSO-d₆) δ (ppm): 2.28 (3H, s, —CH₃), 2.58-2.65 (2H, m, —CH₂), 2.92 (2H, t, —CH₂), 3.65 (3H, s, —OCH₃), 3.74 (3H, s, —OCH₃), 3.93-3.94 (2H, d, —CH₂), 4.01-4.03 (3H, m, —CH & —CH₂), 5.04 (1H, s, —OH), 6.73-6.74 (1H, d, Ar—H), 6.88-6.90 (2H, m, Ar—H), 6.95-7.01 (4H, m, Ar—H), 7.08-7.10 (2H, d, Ar—H), 7.28-7.29 (1H, d, Ar—H), 7.93 (1H, s, ═CH). MS m/z: 498.1 (M + 1). |
| 58 | 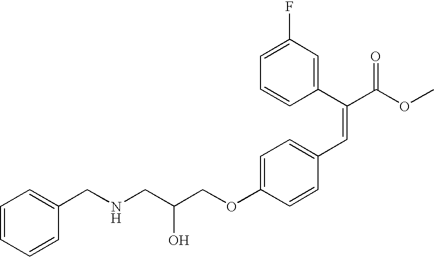 | ¹H NMR (CDCl₃) δ (ppm): 2.72-2.88 (2H, m, —CH₂), 3.78 (3H, s, —OCH₃), 3.81-3.82 (2H, d, —CH₂), 3.92-3.94 (2H, d, —CH₂), 4.00-4.04 (1H, m, —CH), 6.69-6.71 (2H, d, Ar—H), 6.94-7.00 (4H, m, Ar—H), 7.05-7.08 (1H, m, Ar—H), 7.26-7.37 (6H, m, Ar—H), 7.81 (1H, s, ═CH). MS m/z: 436.1 (M + 1). |
| 59 | 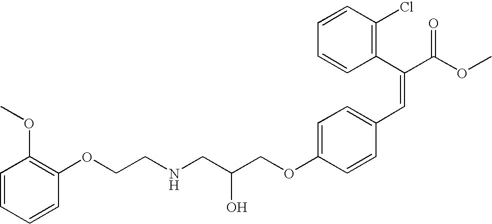 | ¹H NMR (CDCl₃) δ (ppm): 2.79-2.92 (2H, m, —CH₂), 3.04-3.07 (2H, t, —CH₂), 3.77 (3H, s, —OCH₃), 3.82 (3H, s, —OCH₃), 3.93- 3.94 (2H, d, —CH₂), 4.00-4.02 (1H, m, —CH), 4.10-4.13 (2H, t, CH₂), 6.70-6.72 (2H, d, Ar—H), 6.87-6.96 (7H, m, Ar—H), 7.15-7.17 (1H, d, Ar—H), 7.27-7.34 (1H, m, Ar—H), 7.48-7.50 (1H, d, Ar—H), 7.88 (1H, s, ═CH). MS m/z: 512.1 (M + 1). |
| 60 | 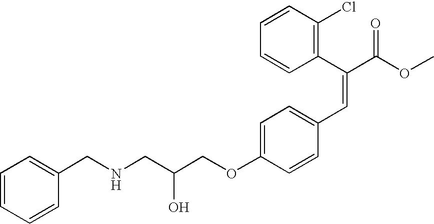 | ¹H NMR (CDCl₃) δ (ppm): 2.72-2.88 (2H, m, —CH₂), 3.77 (3H, s, —OCH₃), 3.81-3.82 (2H, d, —CH₂), 3.91-3.93 (2H, d, —CH₂), 4.02-4.03 (1H, m, —CH), 6.69-6.70 (2H, d, Ar—H), 6.94-6.96 (2H, d, Ar—H), 7.15-7.17 (1H, d, Ar—H), 7.29-7.35 (7H, m, Ar—H), 7.48-7.50 (1H, d, Ar—H), 7.87 (1H, s, ═CH). MS m/z: 452.1 (M + 1). |
| 61 | 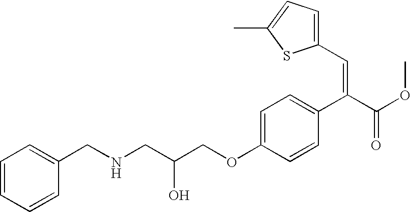 | ¹H NMR (DMSO-d₆) δ (ppm): 2.28 (3H, s, —CH₃), 2.51-2.67 (2H, m, —CH₂), 3.65 (3H, s, —OCH₃), 3.74 (2H, d, —CH₂), 3.92-3.94 (2H, d, —CH₂), 4.03-4.04 (1H, m, —CH), 5.08 (1H, s, —OH), 6.73-6.74 (1H, d, Ar—H), 6.99-7.01 (2H, d, Ar—H), 7.08-7.10 (2H, d, Ar—H), 7.22-7.24 (1H, d, Ar—H), 7.28-7.35 (5H, m, Ar—H), 7.93 (1H, s, ═CH). MS m/z: 437.9 (M + 1). |
| 62 | 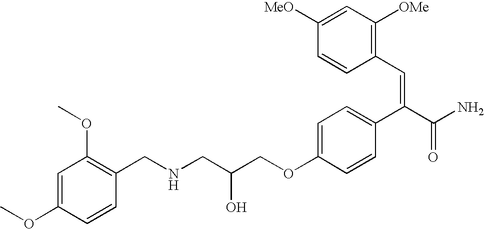 | ¹H NMR (DMSO-d₆) δ (ppm): 2.57-2.63 (2H, m, —CH₂), 3.62-3.63 (2H, d, —CH₂), 3.70 (3H, s, —OCH₃), 3.73 (3H, s, —OCH₃), 3.76 (3H, s, —OCH₃), 3.82 (3H, s, —OCH₃), 3.90-4.00 (3H, m, —CH₂ & —CH), 5.02 (1H, s, —OH), 6.20-6.23 (1H, m, —NH), 6.44-6.47 (1H, m, —NH), 6.50-6.53 (3H, m, Ar—H), 6.59 (1H, s, Ar—H), 6.90-6.92 (2H d, Ar— H), 7.01-7.03 (2H, d, Ar—H), 7.15-7.17 (2H, d, Ar—H), 7.56 (1H, s, ═CH). MS m/z: 522.8 (M + 1). |
| 63 | 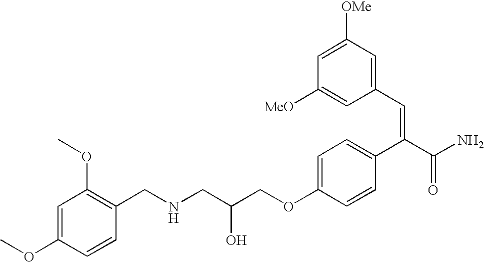 | ¹H NMR (DMSO-d₆) δ (ppm): 2.58-2.67 (2H, m, —CH₂), 3.52 (6H, s, —OCH₃), 3.59- 3.60 (2H, d, —CH₂), 3.74-3.76 (6H, s, —OCH₃), 3.87-3.90 (2H, m, —CH₂), 3.93-3.97 (1H, m, —CH), 5.1 (1H, s, —OH), 6.19 (2H, s, —NH₂), 6.33 (1H, s, Ar—H), 6.46-6.48 (1H, d, Ar—H), 6.53 (1H, s, Ar—H), 6.79 (1H, s, Ar—H), 6.96-6.98 (2H, d, Ar—H), 7.07-7.09 (2H, d, Ar—H), 7.16-7.18 (1H, d, Ar—H), 7.27 (1H, s, Ar—H), 7.33 (1H, s, ═CH). MS m/z: 522.9 (M + 1). |
| 64 | 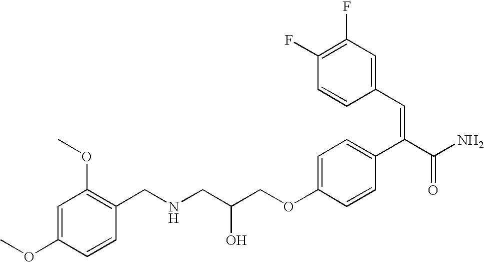 | ¹H NMR (CDCl₃) δ (ppm): 2.73-2.76 (1H, t, —CH), 2.89-2.92 (1H, d, —CH), 3.80-3.83 (8H, m, —OCH₃ & —CH₂), 4.02 (2H, s, —CH₂), 4.14 (1H, s, —CH), 5.50-5.60 (2H, d, —NH₂), 6.44-6.47 (2H, m, Ar—H), 6.73-6.80 (2H, m, Ar—H), 6.93-7.00 (3H, m, Ar—H), 7.12-7.16 (3H, m, Ar—H), 7.73 (1H, s, ═CH). MS m/z: 498.8 (M + 1). |
| 65 | 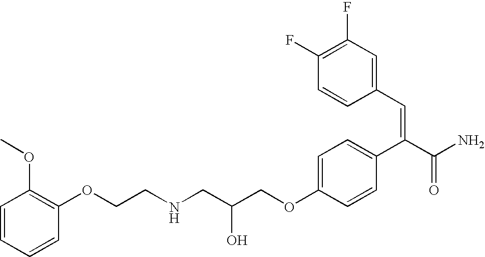 | ¹H NMR (CDCl₃) δ (ppm): 2.86-2.92 (1H, t, —CH), 2.99-3.03 (1H, m, —CH), 3.10-3.13 (2H, t, —CH₂), 3.85 (3H, s, —OCH₃), 4.04- 4.06 (2H, d, —CH₂), 4.14-4.17 (3H, m, —CH₂ & —CH), 5.49 (1H, s, —NH), 5.66 (1H, s, —NH), 6.73-6.78 (1H, m, Ar—H), 6.83 (1H, s, Ar—H), 6.89-7.01 (7H, m, Ar—H), 7.15-7.17 (2H, d, Ar—H), 7.73 (1H, s, ═CH). MS m/z: 498.8 (M + 1). |
| 66 | 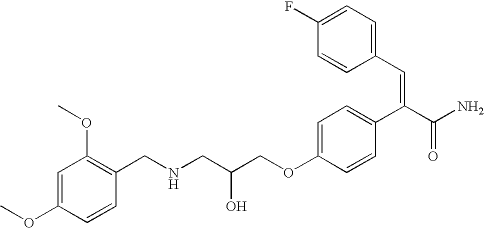 | ¹H NMR (DMSO-d₆) δ (ppm): 2.55-2.58 (1H, t, —CH), 2.62-2.64 (1H, t, —CH), 3.55- 3.57 (2H, d, —CH₂), 3.70 (3H, s, —OCH₃), 3.73 (3H, s, —OCH₃), 3.86-3.89 (2H, t, —CH₂), 3.96-3.97 (1H, d, —CH), 5.02 (1H, s, —OH), 6.44-6.46 (1H, d, Ar—H), 6.52-6.55 (1H, d, Ar—H), 6.80 (1H, s, Ar—H), 6.94- 6.96 (2H, d, Ar—H), 7.01-7.07 (5H, m, Ar— H), 7.15-7.17 (1H, d, Ar—H), 7.27 (2H, s, —NH₂), 7.38 (1H, s, ═CH). MS m/z: 480.9 (M + 1). |
| 67 | 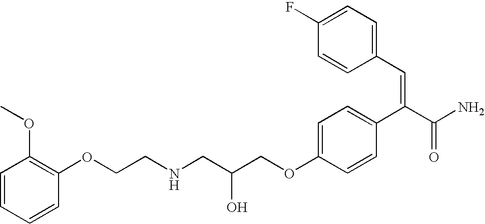 | ¹H NMR (CDCl₃) δ (ppm): 2.91-3.07 (2H, m, —CH₂), 3.15-3.16 (2H, d, —CH₂), 3.86 (3H, s, —OCH₃), 4.05-4.08 (2H, t, —CH₂), 4.18-4.1.9 (3H, m, —CH₂ & —CH), 5.48-5.59 (2H, d, —NH₂), 6.83-6.87 (2H, m, Ar—H), 6.89-7.02 (8H, m, Ar—H), 7.16-7.18 (2H, m, Ar—H), 7.79 (1H, s, ═CH). MS m/z: 480.9 (M + 1). |
| 68 | 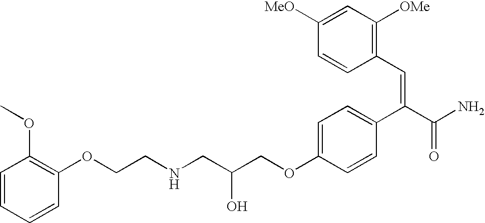 | ¹H NMR (CDCl₃) δ (ppm): 2.87-2.89 (1H, m, —CH), 2.96-2.97 (1H, m, —CH), 3.09-3.11 (2H, t, —CH₂), 3.74 (3H, s, —OCH₃), 3.83- 3.85 (6H, s, —OCH₃), 4.01-4.03 (2H, t, —CH₂), 4.09-4.10 (1H, d, —CH), 4.13-4.16 (2H, t, —CH₂), 5.42 (2H, s, —NH₂), 6.12-6.15 (1H, m, Ar—H), 6.38-6.39 (1H, d, Ar—H), 6.56-6.58 (1H, d, Ar—H), 63.89-6.96 (3H, m, Ar—H), 7.17-7.19 (3H, d, Ar—H), 7.26 (2H, s, Ar—H), 8.10 (1H, s, ═CH). MS m/z: 522.8 (M + 1). |
| 69 | 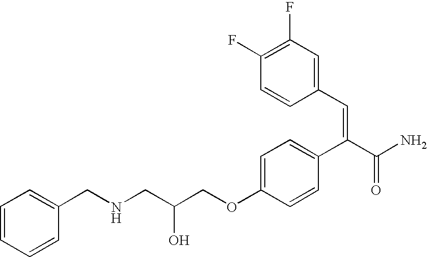 | ¹H NMR (CDCl₃) δ (ppm): 2.80-2.85 (1H, m, —CH), 2.92-2.96 (1H, m, —CH), 3.83-3.90 (2H, m, —CH₂), 4.03-4.04 (2H, d, —CH₂), 4.11 (1H, m, —CH), 5.48-5.52 (2H, d, —NH₂), 6.73-6.78 (1H, m, Ar—H), 6.83 (1H, s, Ar—H), 6.93-7.01 (3H, m, Ar—H), 7.15-7.17 (2H, d, Ar—H), 7.31-7.39 (4H, m, Ar—H), 7.73 (1H, s, ═CH). MS m/z: 438.9 (M + 1). |

### Example 70

### Synthesis of 2-{4-[3-(2-methoxyphenoxyethylamino)propoxy]phenyl}-3-(3,-dimethoxyphenyl)acrylic acid methyl ester

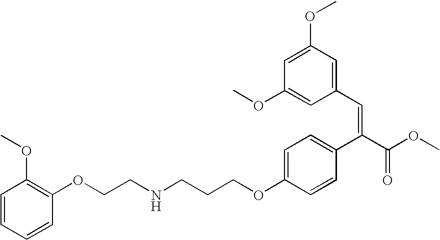

### Step 1: Preparation of 2-[4-(3-bromopropoxy)phenyl]-3-(3,5-dimethoxyphenyl)acrylic acid methyl ester

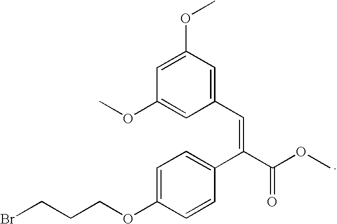

A suspension of 2-[4-hydroxyphenyl]-3-(3,5-dimethoxyphenyl)acrylic acid methyl ester (0.84 g, 2.66 mmol, prepared according to the procedure mentioned for example 1, steps 1-3), 1,3-dibromopropane (0.54 mL, 5.32 mmol) and potassium carbonate (1.1 g, 7.9 mmol) in DMF (8 mL) was stirred for 3 hours. After completion of the reaction, the mixture was diluted with water and extracted with ethyl acetate (2×50 mL). The ethyl acetate layer was washed with water (25 mL×3), dried over anhydrous Na₂SO₄ and concentrated to afford the crude compound (0.56 g, 48% yield).

### Step 2: Synthesis of 2-{4-[3-(2-methoxyphenoxyethylamino)propoxy]phenyl}3-(3,5-dimethoxyphenyl)acrylic acid methyl ester

A suspension of 2-[4-(3-bromopropoxy)phenyl]-3-(3,5-dimethoxyphenyl)acrylic acid methyl ester (0.56 g, 1.3 mmol), 2-(2-methoxyphenoxy)ethylamine (0.23 mL, 1.5 mmol) and potassium carbonate (0.53 g, 3.8 mmol) in DMF (8 mL) was heated to 80° C. for 4 hours. After completion of the reaction, the mixture was diluted with water and extracted with ethyl acetate (2×100 mL). The ethyl acetate layer was washed with water (100 mL×3), dried over anhydrous Na₂SO₄ and concentrated to afford the crude compound, which was purified by column chromatography using 2% methanol in dichloromethane as the eluent, to furnish the title compound (0.05 g, 7.5% yield). ¹H NMR (DMSO-d₆) δ (ppm): 1.86-1.90 (2H, m, —CH₂), 2.72-2.76 (2H, t, —CH₂), 2.87-2.90 (2H, t, —CH₂), 3.53 (6H, s, —OCH₃), 3.70 (3H, s, —OCH₃), 3.73 (3H, s, —OCH₃), 3.99-4.02 (2H, t, —CH₂), 4.04-4.07 (2H, t, —CH₂), 6.27-6.28 (2H, d, Ar—H), 6.38-6.39 (1H, m, Ar—H), 6.87-6.89 (2H, m, Ar—H), 6.94-6.97 (4H, m, Ar—H), 7.07-7.09 (2H, d, Ar—H), 7.67 (1H, s, ═CH). MS m/z: 522.2 (M+1).

The following compounds are prepared according to the procedure given in Example 70

| Ex. No | Structure | Analytical Data |
|---|---|---|
| 71 | 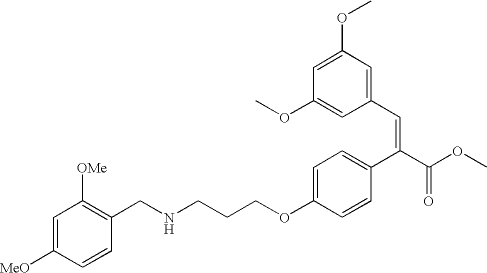 | ¹H NMR (CDCl₃) δ (ppm): 1.95-2.00 (2H, m, —CH₂), 2.75-2.78 (2H, t, —CH₂), 3.54 (6H, s, —OCH₃), 3.74 (2H, s, —CH₂), 3.78-3.79 (9H, s, —OCH₃), 4.02-4.05 (2H, t, —CH₂), 6.24 (2H, s, Ar—H), 6.32 (1H, s, Ar—H), 6.42-6.44 (2H, d, Ar—H), 6.88-6.90 (2H, d, Ar—H), 7.12-7.14 (3H, d, Ar—H), 7.72 (1H, s, ═CH). MS m/z: 521.9 (M + 1). |
| 72 | 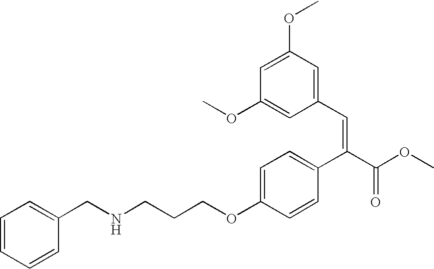 | ¹H NMR (CDCl₃) δ (ppm): 2.14-2.15 (2H, m, —CH₂), 2.94-2.96 (2H, m, —CH₂), 3.59 (6H, s, —OCH₃), 3.83-3.84 (3H, s, —OCH₃), 3.89-3.94 (2H, m, —CH₂), 4.09-4.10 (2H, m, —CH₂), 6.29 (1H, s, Ar—H), 6.36-6.37 (1H, d, Ar—H), 6.91-6.93 (2H, d, Ar—H), 7.16-7.19 (2H, m, Ar—H), 7.26-7.42 (6H, m, Ar—H), 7.73 (1H, s, ═CH). MS m/z: 461.9 (M + 1). |

### Anti-Cancer Experimental Methods

### Anti-Cancer Screen:

The experimental drugs were screened for anti-cancer activity in three cell lines using five concentrations for each compound. The cell lines—HCT 116 (colon), NCIH460 (lung) and U251 (glioma) were maintained in DMEM containing 10% fetal bovine serum. 96-well microtiter plates are inoculated with cells in 100 μL of cell suspension (5×10⁴ cells/mL) for 24 hours at 37° C., 5% CO₂, 95% air and 100% relative humidity. A separate plate with these cell lines is also inoculated to determine cell viability before the addition of the compounds (T₀)

### Addition of Experimental Drugs:

Following 24-hour incubation, test compounds were added to the 96 well plates. Each plate contains one of the above cell lines and the following samples in triplicate: five different dilutions (0.01, 0.1, 1, 10 and 100 μM) of four test compounds, appropriate dilutions of a cytotoxic standard and growth medium (untreated) wells. Test compounds were dissolved in DMSO to prepare 20 mM stock solutions on the day of drug addition and serial dilutions were carried out in complete growth medium at 2× strength, such that 100 μL added to the wells gave final concentrations (0.01, 0.1, 1, 10 and 100 μM) in the well. SAHA was used as the standard drug in these experiments.

### End-Point Measurement:

For T₀ measurement, 24 hours after seeding the cells, 20 μL of 3-(4,5-dimethyl-2-thiazolyl)-2,5-diphenyl-2H-tetrazolium (MTT) solution per well was added to the ‘T₀’ plate and incubated for 3 hours at 37° C. in a CO₂ incubator. The plate containing cells and test compounds was treated similarly after 48 hours of incubation. After 3 hours of MTT addition, well contents were aspirated carefully followed by addition of 150 μL DMSO per well. Plates were agitated to ensure dissolution of the formazan crystals in DMSO and absorbance was read at 570 nm (A₅₇₀).

### Calculation of GI₅₀, TGI and LC₅₀:

Percent growth (PG) is calculated relative to the control and zero measurement wells (T₀) as follows:

PG=(A₅₇₀test−A₅₇₀T₀)/(A₅₇₀control−A₅₇₀T₀)×100 (If A₅₇₀ test>A₅₇₀T₀)

PG=(A₅₇₀test−A₅₇₀T₀)/(A₅₇₀T₀)×100 (If A₅₇₀ test<A₅₇₀T₀),

PG values are plotted against drug concentration to derive the following: GI₅₀ is the concentration required to decrease PG by 50% vs control; TGI is the concentration required to decrease PG by 100% vs control and LC₅₀ is the concentration required to decrease PG by 50% vs T₀. (Mosmann T. Rapid colorimetric assay for cellular growth and survival: application to proliferation and cytotoxicity assays. (J. Immunol. Methods. 1983, 65 (1-2), 55-63; Anne Monks et al); feasibility of high-flux anticancer drug screen using a diverse panel of cultured human tumor cell lines”. (JNCI, Vol. 83, No. 11, 1991). Results for growth inhibition of synthesized compounds are given in Table-1.

### Protocol for Measuring IL-6 in Culture Supernatants

Human lung cancer cell line (NCIH 460) is used for IL-6 measurement. 5000 cells/well were seeded in a 96 well plate and incubated in CO₂ incubator at 37° C. for 24 hours. After 24 hours of incubation the medium from all wells were completely removed and 100 μl of fresh DMEM (Dulbecco's Modified Eagle's Medium) containing 10% FCS was added. 100 μl of different concentrations of the drug were added and incubated for 5 hours in a CO₂ incubator at 37° C. After 5 hours of incubation the supernatants were collected and stored at −80° C. The concentration of IL-6 in the supernatant was measured using an ELISA kit and the IC₅₀ value of the compound was determined from the dose response curve. Table 1 gives the anticancer activity and Il-6 inhibition data for the selected compounds.

TABLE 1

| | | IL-6 % Inhibition at |
|---|---|---|
| Ex. No | Mean GI₅₀(μM) | 10 μM |
| 3 | 4.1 | 67 |
| 4 | 32.4 | ND |
| 5 | 3.1 | 37 |
| 6 | 5.2 | 73 |
| 7 | 2.2 | 58 |
| 8 | 3.6 | 44 |
| 9 | >100 | 34 |
| 10 | 35.3 | 4.5 |
| 11 | 37.3 | 29 |
| 12 | 3 | 55 |
| 13 | 3.5 | 67 |
| 14 | 4 | 38 |
| 15 | 2.8 | 44 |
| 16 | 3.1 | 68 |
| 17 | 3.4 | 25 |
| 18 | 5.3 | 25 |
| 19 | 4.9 | 15.9 |
| 20 | 16.8 | 0 |
| 21 | 13.1 | 22.8 |
| 22 | 16.1 | 59.8 |
| 23 | 11.7 | 54.2 |
| 24 | 15.5 | 0 |
| 25 | 27.8 | 20.5 |
| 26 | 12.1 | 36.2 |
| 27 | 6.5 | 32.7 |
| 28 | 1.3 | 78.5 |
| 29 | 1.9 | 64.2 |
| 30 | 1.4 | 27.8 |
| 31 | 0.9 | 2.1 |
| 32 | 0.6 | 70.5 |
| 33 | 2.6 | 61.2 |
| 34 | 18.7 | 41.9 |
| 35 | 5.1 | 72.7 |
| 36 | 12.0 | 27.9 |
| 37 | 6.2 | 68.1 |
| 38 | 9.0 | 69.1 |
| 39 | 6.3 | 41.8 |
| 40 | 9.7 | 70.5 |
| 41 | 11.4 | 68 |
| 42 | 12 | 13 |
| 43 | 0.86 | 9.6 |
| 44 | 2.1 | 7.7 |
| 45 | 1.2 | 56 |
| 46 | 1.6 | 48 |
| 47 | 1.3 | 59 |
| 48 | 1.6 | 9 |
| 49 | 2.1 | 56 |

Inhibition of pSTAT3

The experimental drugs were tested for their effect on phosphorylation of STAT3 in HEPG2 (liver cancer) cells induced by IL-6. Test compounds were dissolved in DMSO at 20 mM and diluted in a growth medium at 2× strength. Final DMSO concentration was less than 0.5%. 100 μL of cell suspension (10×10⁴ cells/mL) was added to the wells of 96 well plates and allowed to adhere for 24 hours in a CO₂ incubator at 37° C. Following overnight incubation, the culture medium was replaced with 100 μL of fresh growth medium and 100 μL of test compound (s), standard (Curcumin) and growth medium (untreated) wells. The plates were incubated in CO₂ incubator at 37° C. for 4 hours. After compound treatment, IL-6 (10 ng/mL) was added to the wells and incubated for 30 minutes to induce pSTAT3.

At the end of the 30 minutes incubation, cells were rinsed with ice-cold PBS and lysed with 1× Cell lysis buffer containing 1 mM PMSF (provided in the kit) on ice for 5 minutes. The cells were scraped off and transferred to an appropriate tube, sonicated on ice and centrifuged for 10 minutes at 4° C. The supernatant (cell lysate) was collected and stored at −80° C. and the levels of pSTAT3 were measured in the cell lysate by Path Scan Phospho-STAT3 (Tyr705) Sandwich ELISA kit (Cell Signaling, Cat No. 7300) by following the kit instructions. Results for pSTAT3 inhibition are given in Table-2.

TABLE 2
Inhibition of pSTAT3 by test compounds

| | % pSTAT3 Inhibition |
|---|---|
| Ex. No | (at 10 μM) |
| 1 | 22.2 |
| 35 | 32.7 |
| 37 | 42.0 |
| 38 | 77.2 |
| 40 | 71.0 |
| 43 | 46.4 |
| 49 | 58.4 |
| 51 | 49.9 |
| 53 | 61.4 |
| 56 | 56.2 |
| 57 | 66.5 |
| 64 | 32.2 |
| 65 | 0 |

1. A compound of formula (I), 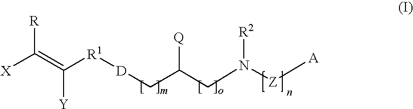
their derivatives, analogs, tautomeric forms, stereoisomers which includes both geometrical and optical isomers, polymorphs, solvates, intermediates, pharmaceutically acceptable salts, pharmaceutical compositions, metabolites and prodrugs thereof; wherein, the configuration around the double bonds may be E/Z and a mixture;
R and R¹ may be same or different and independently represent optionally substituted groups selected from cycloalkyl, aryl, arylalkyl, arylalkenyl, arylalkynyl, heterocyclyl, heteroaryl, heteroarylalkyl, heteroarylalkenyl and heteroarylalkynyl; R² represents H, optionally substituted groups selected from alkyl, cycloalkyl, aryl and arylalkyl;
X and Y independently represent H; optionally substituted groups selected from alkyl, wherein when one of X or Y is hydrogen or unsubstituted alkyl, the other is neither of hydrogen nor of unsubstituted alkyl; —COOR³; —CONR³R⁴; —CN; —CH₂NR³R⁴; —CH₂CH₂NR³R⁴; —CH₂OR³; —CH₂CH₂OR³; —CH₂OCONR³R⁴ and —CH₂NR³COR⁴; wherein R³ and R⁴ may be same or different and independently represent hydrogen; optionally substituted groups selected from alkyl; alkoxy; alkenyl; alkynyl; cycloalkyl; aryl; arylalkyl; arylalkenyl; arylalkynyl; heterocyclyl; heteroaryl; heteroarylalkyl; heteroarylalkenyl and heteroarylalkynyl or R³ and R⁴ combine to form a ring structure;
D represents —O— or —CH₂—; Q represents H or OR⁵; wherein R⁵ represents H, optionally substituted groups selected from alkyl, cycloalkyl, aryl, heterocyclyl and heteroaryl; Z represents —CH₂— or —CO—; A represents optionally substituted groups selected from aryl, arylalkyl, aryloxy, heterocyclyl and heteroaryl; wherein m, n and o are integers ranging from 0 to 5 and they may be same or different;
when the groups R, R¹, R², R³, R⁴, R⁵ and A are substituted, the substituents which may be one or more are selected from halogens comprising fluorine, chlorine, bromine and iodine; hydroxy; nitro; cyano; oxo (═O); thioxo (═S); azido; nitroso; amino; hydrazino; formyl; alkyl; alkoxy; aryl; haloalkyl group comprising trifluoromethyl, tribromomethyl and trichloromethyl; haloalkoxy group comprising —OCH₂Cl, —OCHF₂ and —OCF₃; arylalkoxy group comprising benzyloxy and phenylethoxy; cycloalkyl; —O-cycloalkyl; aryl; alkoxy; heterocyclyl; heteroaryl; alkylamino; —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a})SOR^{b}; —N(R^{a})SO₂R^{b}; —NR^{a}C(O)OR^{b}; —NR^{a}R^{b}; —NR^{a}C(O)R^{b}—; NR^{a}C(S)R^{b}—; —SONR^{a}R^{b}—; —SO₂NR^{a}R^{b}—; —OR^{a}; —OR^{a}C(O)OR^{b}—; —OC(O)NR^{a}R^{b}; OC(O)R^{a}; —OC(O)NR^{a}R^{b}—; —R^{a}NR^{b}R^{c}; —R^{a}OR^{b}—; —SR^{a}; —SOR^{a} and —SO₂R^{a}; R^{a}, R^{b} and R^{c} each independently represents hydrogen atom; substituted or unsubstituted groups selected from alkyl; aryl; arylalkyl; cycloalkyl; heterocyclyl; heteroaryl, heteroarylalkyl and R^{a}, R^{b} and R^{c} combine to form 3-7 membered ring having 0-2 hetero atoms;
the substituents are in turn are further substituted by halogens comprising fluorine, chlorine, bromine and iodine; hydroxy; nitro; cycloalkyl; cyano; azido; nitroso, amino, hydrazino, formyl; alkyl and haloalkyl groups comprising trifluoromethyl and tribromoethyl.

2. A compound according to claim 1 wherein, R and R¹ may be same or different and independently represent optionally substituted groups selected from cycloalkyl group comprising cyclopropyl, cyclobutyl, cyclopentyl, cyclohexyl, cyclooctanyl, perhydronaphthyl, adamantyl, norbornyl and spiro[4.4]-non-2-yl; aryl group comprising phenyl, naphthyl, biphenyl and indanyl; arylalkyl group comprising benzyl and phenylethyl; arylalkenyl group comprising phenylethenyl and phenylpropenyl; arylalkynyl group comprising phenylethynyl and phenylpropynyl; heterocyclyl group comprising azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, decahydroisoquinolyl, benzimidazolyl, thiadiazolyl, benzopyranyl, benzothiazolyl, benzooxazolyl, thienyl, morpholinyl, thiomorpholinyl, thiamorpholinyl sulfoxide, furyl, tetrahydrofuryl, tetrahydropyranyl, chromanyl and isochromanyl; heteroaryl group; heteroarylalkyl group comprising thienylpropyl, pyridinylethyl and indolylpropyl; heteroarylalkenyl group comprising thienylpropenyl, pyridinylethenyl and indolylpropenyl; heteroarylalkynyl group comprising thienylpropynyl, pyridinylethynyl and indolylpropynyl;
X and Y independently represent H; optionally substituted groups selected from alkyl group comprising methyl, ethyl, n-propyl, isopropyl, butyl, isobutyl, t-butyl, pentyl, hexyl, heptyl and octyl, wherein when one of X or Y is hydrogen or unsubstituted alkyl, the other is neither of hydrogen nor of unsubstituted alkyl; —COOR³; —CONR³R⁴; —CN; —CH₂NR³R⁴; —CH₂OR³; —CH₂CH₂OR³; —CH₂OCONR³R⁴ and —CH₂NR³COR⁴; wherein R³ and R⁴ may be same or different and independently represent hydrogen; optionally substituted groups selected from alkyl; alkoxy group comprising —OCH₃ and —OC₂H₅; alkenyl group comprising ethenyl, 1-propenyl, 2-propenyl, iso-propenyl, 2-methyl-1-propenyl, 1-butenyl and 2-butenyl; alkynyl group comprising ethynyl, propynyl and butynyl; cycloalkyl; aryl; arylalkyl; arylalkenyl; arylalkynyl; heterocyclyl; heteroaryl; heteroarylalkyl; heteroarylalkenyl and heteroarylalkynyl or R³ and R⁴ combine to form a ring structure;
D represents —O— or —CH₂—; Q represents H or OR⁵; wherein R⁵ represents H, optionally substituted groups selected from alkyl, cycloalkyl, aryl and heteroaryl; Z represents —CH₂— or —CO—; A represents optionally substituted groups selected from aryl, arylalkyl, aryloxy group comprising —O-phenyl, —O-biphenyl; heterocyclyl and heteroaryl.

3. The compound according to claim 1 selected from the compounds consisting of:
2-{4-[3-(2,4-Dimethoxybenzylamino)-2-hydroxypropoxy]phenyl}-3-(4-fluoro-3-methylphenyl)acrylic acid methyl ester;
2-{4-[3-(2,4-Dimethoxybenzylamino)-2-hydroxypropoxyl]phenyl}-3-(4-fluorophenyl)acrylic acid methyl ester;
2-{4-[3-(2,4-Dimethoxybenzylamino)-2-hydroxypropoxyl]phenyl}-3-(3,5-dimethoxy phenyl)acrylic acid methyl ester;
2-{4-[3-(4-Trifluoromethoxybenzylamino)-2-hydroxypropoxyl]phenyl}-3-(3,5-dimethoxyphenyl)acrylic acid methyl ester;
2-{4-[3-(2-Methoxyphenoxyethylamino)-2-hydroxypropoxyl]phenyl}-3-(3,5-dimethoxyphenyl)acrylic acid methyl ester;
2-{4-[3-(2,4-Dimethoxy benzylamino)-2-hydroxypropoxyl]phenyl}-3-(3,4,5-trimethoxyphenyl)acrylic acid methyl ester;
2-{4-[3-(2-Methoxyphenoxyethylamino)-2-hydroxypropoxyl]phenyl}-3-(4-fluoro-3-methoxyphenyl)acrylic acid methyl ester;
2-{4-[3-(4-Difluoromethoxybenzylamino)-2-hydroxypropoxyl]phenyl}-3-(3,4,5-trimethoxyphenyl)acrylic acid methyl ester;
2-{4-[3-(2,4-Dimethoxybenzylamino)-2-hydroxypropoxyl]phenyl}-3-(4-fluoro-3-methoxyphenyl)acrylic acid methyl ester;
3-{4-[3-(4-Fluorobenzylamino)-2-hydroxypropoxyl]phenyl}-2-phenylacrylic acid methyl ester;
2-{4-[3-(2-Methoxyphenoxyethylamino)-2-hydroxypropoxyl]phenyl}-3-(3,4,5-trimethoxyphenyl)acrylic acid methyl ester;
3-{4-[3-(2,4-Dimethoxybenzylamino)-2-hydroxypropoxyl]phenyl}-2-(4-methoxy phenyl)acrylic acid methyl ester;
2-{4-[3-(2,4-Dimethoxy benzylamino)-2-hydroxypropoxyl]phenyl}-3-(3,4,-dimethoxyphenyl)acrylic acid methyl ester;
2-{4-[3-(2-Methoxyphenoxyethylamino)-2-hydroxypropoxyl]phenyl}-3-(3,4,-dimethoxyphenyl)acrylic acid methyl ester;
2-{4-[3-(2,4-Dimethoxybenzylamino)-2-hydroxypropoxyl]phenyl}-3-(3,4-difluorophenyl)acrylic acid methyl ester;
3-{4-[3-(2,4-Dimethoxybenzylamino)-2-hydroxypropoxyl]phenyl}-2-phenylacrylic acid methyl ester;
2-{4-[3-(2,4-Dimethoxybenzylamino)-2-hydroxypropoxy]phenyl}-3-phenylacrylic acid methyl ester;
2-{4-[3-(Benzylamino)-2-hydroxypropoxy]phenyl}-3-(3,5-dimethoxyphenyl)acrylic acid methyl ester;
2-{4-[3-(2-Methoxyphenoxyethylamino)-2-hydroxypropoxy]phenyl}-3-(4-fluoro-3-methylphenyl)acrylic acid methyl ester;
2-{4-[3-(4-Fluorobenzylamino)-2-hydroxypropoxy]phenyl}-3-(3,5-dimethoxy phenyl)acrylic acid methyl ester;
2-{4-[3-(Benzylamino)-2-hydroxypropoxy]phenyl}-3-(4-fluoro-3-methylphenyl)acrylic acid methyl ester;
2-{4-[3-(2,4-Dimethoxybenzylamino)-2-hydroxypropoxy]phenyl}-3-(4-chlorophenyl)-acrylic acid methyl ester;
2-{4-[3-(2-Methoxyphenoxyethylamino)-2-hydroxypropoxy]phenyl}-3-(4-(methylthio)phenyl)acrylic acid methyl ester;
2-{4-[3-(Benzylamino)-2-hydroxypropoxy]phenyl}-3-phenylacrylic acid methyl ester;
2-{4-[3-(2-Methoxyphenoxyethylamino)-2-hydroxypropoxy]phenyl}-3-phenylacrylic acid methyl ester;
2-{4-[3-(2-Methoxyphenoxyethylamino)-2-hydroxypropoxy]phenyl}-3-(4-fluorophenyl)acrylic acid methyl ester;
2-{4-[3-(Benzylamino)-2-hydroxypropoxy]phenyl}-3-(4-chlorophenyl)acrylic acid methyl ester;
2-{4-[3-(2,4-Dimethoxybenzylamino)-2-hydroxypropoxy]phenyl}-3-(4-(methylthio) phenyl)acrylic acid methyl ester;
3-{4-[3-(2-Methoxyphenoxyethylamino)-2-hydroxypropoxy]phenyl}-2-(4-(methoxy)phenyl)acrylic acid methyl ester;
3-{4-[3-(Benzylamino)-2-hydroxypropoxy]phenyl}-2-(4-(methoxy)phenyl)acrylic acid methyl ester;
3-{-4-[3-(Benzylamino)-2-hydroxypropoxy]phenyl}-2-(thiophen-2-yl)acrylic acid methyl ester;
2-{4-[3-(2,4-Dimethoxybenzylamino)-2-hydroxypropoxy]phenyl}-3-(2,4-dimethoxy phenyl)acrylic acid methyl ester;
2-{4-[3-(2-Methoxyphenoxyethylamino)-2-hydroxypropoxy]phenyl}-3-(2,4-dimethoxy phenyl)acrylic acid methyl ester;
3-{4-[3-(2-Methoxyphenoxyethylamino)-2-hydroxypropoxy]phenyl}-2-(thiophen-2-yl)acrylic acid methyl ester;
3-{4-[3-(2,4-Dimethoxybenzylamino)-2-hydroxypropoxy]phenyl}-2-(4-fluorophenyl)acrylic acid methyl ester;
3-{4-[3-(Benzylamino)-2-hydroxypropoxy]phenyl}-2-(4-fluorophenyl)acrylic acid methyl ester;
3-{4-[3-(2,4-Dimethoxybenzylamino)-2-hydroxypropoxy]phenyl}-2-(4-methylphenyl)acrylic acid methyl ester;
3-{4-[3-(2-Methoxyphenoxyethylamino)-2-hydroxypropoxy]phenyl}-2-(4-methylphenyl)acrylic acid methyl ester;
3-{4-[3-(Benzylamino)-2-hydroxypropoxy]phenyl}-2-(4-methylphenyl)acrylic acid methyl ester;
3-{4-[3-(2,4-Dimethoxybenzylamino)-2-hydroxypropoxy]phenyl}-2-(thiophen-2-yl)acrylic acid methyl ester;
3-{4-[3-(2-Methoxyphenoxyethylamino)-2-hydroxypropoxy]phenyl}-2-(4-fluorophenyl)acrylic acid methyl ester;
3-{4-[3-(2-Methoxyphenoxyethylamino)-2-hydroxypropoxy]phenyl}-2-phenylacrylic acid methyl ester;
3-{2[3-(Benzylamino)-2-hydroxypropoxy]phenyl}-2-(4-methoxyphenyl)acrylic acid methyl ester;
3-{4-[3-(Benzylamino)-2-hydroxypropoxy]phenyl}-2-phenylacrylic acid methyl ester;
3-{2-[3-(2,4-Dimethoxybenzylamino)-2-hydroxypropoxy]phenyl}-2-(4-methoxy phenyl)acrylic acid methyl ester;
3-{2-[3-(2-Methoxyphenoxyethylamino)-2-hydroxypropoxy]phenyl}-2-(4-methoxyphenyl)acrylic acid methyl ester;
3-{4-[3-(2,4-Dimethoxybenzylamino)-2-hydroxypropoxy]phenyl}-2-(3-chloro phenyl)acrylic acid methyl ester;
3-{4-[3-(2-Methoxyphenoxyethylamino)-2-hydroxypropoxy]phenyl}-2-(3-chloro phenyl)acrylic acid methyl ester;
3-{4-[3-(Benzylamino)-2-hydroxypropoxy]phenyl}-2-(3-chlorophenyl)acrylic acid methyl ester;
3-{4-[3-(2,4-Dimethoxybenzylamino)-2-hydroxypropoxy]phenyl}-2-(3-fluoro phenyl)acrylic acid methyl ester;
3-{4-[3-(2-Methoxyphenoxyethylamino)-2-hydroxypropoxy]phenyl}-2-(3-fluoro phenyl)acrylic acid methyl ester;
3-{4-[3-(2,4-Dimethoxybenzylamino)-2-hydroxypropoxy]phenyl}-2-(2-chlorophenyl)acrylic acid methyl ester
3-{4-[3-(2,4-Dimethoxybenzylamino)-2-hydroxypropoxy]phenyl}-2-(2-fluoro phenyl)acrylic acid methyl ester;
3-{4-[3-(2-Methoxyphenoxyethylamino)-2-hydroxypropoxy]phenyl}-2-(2-fluoro phenyl)acrylic acid methyl ester;
3-{4-[3-(Benzylamino)-2-hydroxypropoxy]phenyl}-2-(2-fluorophenyl)acrylic acid methyl ester;
2-{4-[3-(2,4-Dimethoxybenzylamino)-2-hydroxypropoxy]phenyl}-3-(5-m ethyl-thiophen-2-yl)acrylic acid methyl ester;
2-{4-[3-(2-Methoxyphenoxyethylamino)-2-hydroxypropoxy]phenyl}-3-(5-methyl-thiophen-2-yl)acrylic acid methyl ester;
3-{4-[3-(Benzylamino)-2-hydroxypropoxy]phenyl}-2-(3-fluorophenyl)acrylic acid methyl ester;
3-{4-[3-(2-Methoxyphenoxyethylamino)-2-hydroxypropoxy]phenyl}-2-(2-chloro phenyl)acrylic acid methyl ester;
3-{4-[3-(Benzylamino)-2-hydroxypropoxy]phenyl}-2-(2-chlorophenyl)acrylic acid methyl ester;
2-{4-[3-(Benzylamino)-2-hydroxypropoxy]phenyl}-3-(5-methylthiophen-2-yl)acrylic acid methyl ester;
2-{4-[3-(2,4-Dimethoxybenzylamino)-2-hydroxypropoxy]phenyl}-3-(2,4-dimethoxyphenyl)acrylamide;
2-{4-[3-(2,4-Dimethoxybenzylamino)-2-hydroxypropoxy]phenyl}-3-(3,5-dimethoxyphenyl)acrylamide;
2-{4-[3-(2,4-Dimethoxybenzylamino)-2-hydroxypropoxy]phenyl}-3-(3,4-difluorophenyl)acrylamide;
2-{4-[3-(2-Methoxyphenoxyethylamino)-2-hydroxypropoxy]phenyl}-3-(3,4-difluorophenyl)acrylamide;
2-{4-[3-(2,4-Dimethoxybenzylamino)-2-hydroxypropoxy]phenyl}-3-(4-fluorophenyl)acrylamide;
2-{4-[3-(2-Methoxyphenoxyethylamino)-2-hydroxypropoxy]phenyl}-3-(4-fluorophenyl)acrylamide;
2-{4-[3-(2-Methoxyphenoxyethylamino)-2-hydroxypropoxy]phenyl}-3-(2,4-dimethoxy phenyl)acrylamide;
2-{4-[3-(Benzylamino)-2-hydroxypropoxy]phenyl}-3-(3,4-difluorophenyl) acrylamide;
2-{4-[3-(2-Methoxyphenoxyethylamino)propoxy]phenyl}-3-(3,5-dimethoxyphenyl)acrylic acid methyl ester;
2-{4-[3-(2,4-dimethoxybenzylamino)propoxy]phenyl}-3-(3,5-dimethoxyphenyl)acrylic acid methyl ester and
2-{4-[3-(Benzylamino)propoxy]phenyl}-3-(3,5-dimethoxyphenyl)acrylic acid methyl ester.

4. A process for the preparation of compound of formula (I) according to claim 1, comprising condensing the compound of formula (Ie) or the compound of formula (Id) with the amino compound (If), wherein all the groups R, R¹, R², A, D, X, Y, Q, Z, L, m, n and o are as defined earlier and when one of X or Y is hydrogen or unsubstituted alkyl, the other is neither of hydrogen nor of unsubstituted alkyl.

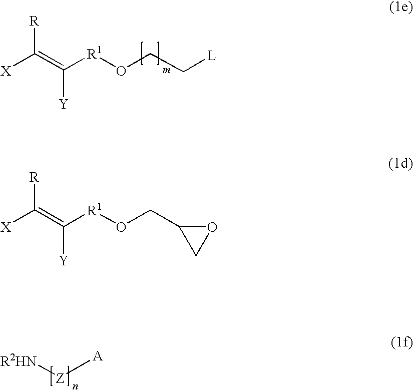

5. A pharmaceutical composition comprising a compound of formula (I) according to claim 1 as an active ingredient, along with a pharmaceutically acceptable carrier, diluent, excipient or solvate.

6. A pharmaceutical composition according to claim 5, wherein the composition is in the form of a tablet, capsule, powder, syrup, solution, aerosol or suspension.

7. A method of lowering plasma concentrations of IL-6, comprising administering an effective amount of a compound according to claim 1, to the mammal in need thereof.

8. A method of treating inflammatory diseases and cancer, particularly those mediated by cytokines selected from IL-6, through pSTAT3 inhibition, comprising administering an effective amount of a compound according to claim 1, to the mammal in need thereof.

9. A method of prophylaxis or treatment of autoimmune diseases, neurological disorders, inflammatory diseases selected from rheumatoid arthritis, psoriasis, glomerulonephritis, Castleman's disease; bone loss, hypercalcemia, edema; septic shock, toxic shock, multiple myeloma, pancreatitis, proliferative conditions or cancer, cancer cachexia; osteoporosis, anemia, leukemia, diabetes, liver diseases, inflammatory bowel disease, neuropathy, nephritis, Kaposis's sarcoma, hepatitis, colitis, endometriosis and infection in a mammal comprising administering an effective amount of a compound according to claim 1, to the mammal in need thereof.

10. A method for the treatment of cancer, either as a monotherapy in those cancer cells, which have constitutively active pSTAT3 by administering a compound according to claim 1, to a mammal in need thereof or as a combination therapy by administering these pSTAT3 inhibitors, along with other clinically relevant cytotoxic agents or non-cytotoxic agents.

11. A pharmaceutical composition comprising a compound of formula (I), according to claim 3, as an active ingredient, along with a pharmaceutically acceptable carrier, diluent, excipient or solvate.

12. A method of lowering plasma concentrations of IL-6, comprising administering an effective amount of a compound according to claim 3, to the mammal in need thereof.

13. A method of treating inflammatory diseases and cancer, particularly those mediated by cytokines selected from IL-6, through pSTAT3 inhibition, comprising administering an effective amount of a compound according to claim 3, to the mammal in need thereof.

14. A method of prophylaxis or treatment of autoimmune diseases, neurological disorders, inflammatory diseases selected from rheumatoid arthritis, psoriasis, glomerulonephritis, Castleman's disease; bone loss, hypercalcemia, edema; septic shock, toxic shock, multiple myeloma, pancreatitis, proliferative conditions or cancer, cancer cachexia; osteoporosis, anemia, leukemia, diabetes, liver diseases, inflammatory bowel disease, neuropathy, nephritis, Kaposis's sarcoma, hepatitis, colitis, endometriosis and infection in a mammal comprising administering an effective amount of a compound according to claim 3, to the mammal in need thereof.

15. A method for the treatment of cancer, either as a monotherapy in those cancer cells, which have constitutively active pSTAT3 by administering a compound according to claim 3, to a mammal in need thereof or as a combination therapy by administering these pSTAT3 inhibitors, along with other clinically relevant cytotoxic agents or non-cytotoxic agents.
